(19) United States
(12) Patent Application Publication
(10) Pub. No.: US 2020/0095547 A1
(43) Pub. Date: Mar. 26, 2020

(54) METHODS FOR MANUFACTURING T CELLS EXPRESSING OF CHIMERIC ANTIGEN RECEPTORS AND OTHER RECEPTORS

(71) Applicants: Darya ALIZADEH, Duarte, CA (US); Christine BROWN, Duarte, CA (US); Stephen J. FORMAN, Duarte, CA (US); City of Hope, Duarte, CA (US)

(72) Inventors: Darya Alizadeh, Duarte, CA (US); Christine E. Brown, Duarte, CA (US); Stephen J. Forman, Duarte, CA (US)

(21) Appl. No.: 16/465,847

(22) PCT Filed: Dec. 1, 2017
(86) PCT No.: PCT/US2017/064326

§ 371 Date: May 31, 2019

Related U.S. Application Data
(60) Provisional application No. 62/429,665, filed on Dec. 2, 2016.

(51) Int. Cl. C12N 5/0783

(52) U.S. Cl. CPC C12N 5/0636; C12N 2501/2302; C12N 2501/2307; C12N 2501/599; C12N 2501/2321; C12N 2501/515; C12N 2501/505; C12N 2501/2315

(57) ABSTRACT

A method for preparing T cell populations for use in CAR T cell therapy and other immune cell therapies is described.

### STATEMENT REGARDING FEDERALLY SPONSORED RESEARCH

This invention is made with government support in the ______. The United States government has certain rights in the invention.

### BACKGROUND

Adoptive T cell therapy (ACT) utilizing ex vivo expanded autologous and allogeneic T cells is an attractive therapeutic approach for the treatment of viral infection, cancer and autoimmune disease. Methods that enable the rapid generation of large numbers of therapeutic T cells are critical to the potency and safety of ACT. Various T cell enrichment methods, including selection of defined T cell subsets, as well as expansion methods have been used for ACT. It is desirable to employ a T cell population that permits relatively high activity in vivo and relatively high proliferation potential.

### SUMMARY

Described herein is a method for manufacturing T cell populations useful in T cell therapy, for example, T cells expressing a recombinant T cell receptor (e.g., a chimeric antigen receptor (“CAR”) or T cell receptor (“TCR”)) or tumor infiltrating lymphocytes (“TIL”). The T cell populations are also useful for a variety of purposes requiring a highly active, long-lived T cell population. The methods described herein entail expanding T cell populations in the presence of exogenously added IL-15 and in presence of minimal or no exogenously added IL-2 (e.g., less than 50 U/ml, less than 40 U/ml, less than 30 U/ml, less than 20 U/ml, less than 10 U/ml, less than 5 U/ml or even less than 1 U/ml). In some cases, the cells are expanded in the presence of exogenously added IL-15 (e.g., at least 10 ng/ml) and minimal or no exogenously added IL-2 (e.g., less than 50 U/ml, less than 40 U/ml, less than 30 U/ml, less than 20 U/ml, less than 10 U/ml, less than 5 U/ml or even less than 1 U/ml) and minimal or no exogenously added IL-7 (e.g., less than 10 ng/ml, less than 8 ng/ml, less than 6 ng/ml, less than 5 ng/ml, less than 3 ng/ml or even less than 1 ng/ml). In some cases, the cells are expanded in the presence of exogenously added IL-15 (e.g., at least 10 ng/ml) and minimal or no exogenously added IL-2 (e.g., less than 50 U/ml, less than 40 U/ml, less than 30 U/ml, less than 20 U/ml, less than 10 U/ml, less than 5 U/ml or even less than 1 U/ml), minimal or no exogenously added IL-7 (e.g., less than 10 ng/ml, less than 8 ng/ml, less than 6 ng/ml, less than 5 ng/ml, less than 3 ng/ml or even less than 1 ng/ml) and minimal or no exogenously added IL-21 (e.g., less than 10 ng/ml, less than 8 ng/ml, less than 6 ng/ml, less than 5 ng/ml, less than 3 ng/ml or even less than 1 ng/ml). In some cases, the only exogenously added interleukin is IL-15 (preferably human IL-15). In some cases, all exogenously added interleukins other than IL-15 (e.g., IL-7, IL-21, IL-4 and IL-9) are present at less than 10 ng/ml (less than 8 ng/ml, less than 6 ng/ml, less than 5 ng/ml, 3 ng/ml or even less than 1 ng/ml) and exogenously added IL-2 is present at less than 50 U/ml (less than 40 U/ml, less than 30 U/ml, less than 20 U/ml, less than 10 U/ml, less than 5 U/ml or even less than 1 U/ml). Exogenously added interleukins are those that are added to the culture media as opposed to being generated by the cells themselves.

The T cell populations that can be expanded using the manufacturing methods described herein can include: naïve T cells (T_{N}), memory stem cells (T_{SCM}), central memory T cells (T_{CM}) and combinations thereof in addition to other cells such as effector T cells (T_{E}) or effector memory T cells (T_{EM}). FIG. 1 schematically depicts these cells type and certain of the cell surface markers expressed by each. T cell populations that are primarily naïve T cells (T_{N}), memory stem cells (T_{SCM}), and central memory T cells (T_{CM}) with few T_{E} and T_{EM} cells can be described as T_{CM/SCM/N} cells or T_{CM/SCM/N} cell populations. These cell populations can be derived from peripheral blood mononuclear cells (PBMC) by both: 1) depleting unwanted cell populations such as CD14 expressing myeloid cells and CD25 expressing cells; and 2) enriching for CD62L expressing memory and naïve T cells. Thus, the resulting population of cells includes T naïve (T_{N}) and stem memory cells (T_{SCM}) expressing CD45RA and CD62L. It also includes the population of central memory T cells (T_{CM}) that express CD45RO and CD62L. T_{CM/SCM/N} cell populations differ from previously described T_{CM} cell populations in that their preparation does not entail depletion of CD45RA+T cells. T_{CM/SCM/N} cell populations, upon preparation, are relatively free of effector memory cells (T_{EM}) and effector cells (T_{E}). In addition, such T cell populations have a relatively high proportion or CD45RA+CD45RO− T cells.

In certain embodiments of the manufacturing method described herein, a population of T cells, e.g., a T_{CM/SCM/N} cell population, a T_{CM} cell population, a T_{N} or unselected PBMC, is stimulated and transduced with a vector expressing a desired T cell receptor, e.g., a CAR. After transduction, the cells are expanded by culturing in a medium comprising exogenously added IL-15 at greater than or equal to 5 ng/ml or 10 ng/ml and exogenously added IL-2 at less than or equal to 50, 40, 30, 20 or 10 U/ml (“High IL-15/Low IL-2 culture conditions”). In some cases, exogenously added IL-7 and or exogenously added IL-21 are each present at less than 10 ng/ml (less than 5 ng/ml, 3 ng/ml or 1 ng/ml or there is no exogenously added IL-2, IL-7 or IL-21). As the T cells are expanded over a period of days, differentiation will occur giving rise to, for example, additional T_{E} cells and additional T_{EM} cells. Thus, where the starting T cell population is a T_{CM/SCM/N} cell population, culturing will, over time lead to an increase in the proportion of CD45RA+CD45RO+T cells and the proportion of CD45RA-CD45RO+T cells. However, compared to certain conventional culture conditions with relatively low exogenously added IL-15 and relatively high exogenously added IL-2, the High IL-15/Low IL-2 culture conditions described herein result in a higher proportion of desirable CD45RA+CD45RO− T cells. In addition, as demonstrated herein, T cell populations expressing a CAR expanded under the High IL-15/Low IL-2 culture conditions express a lower level of exhaustion markers such as 2B4 and Lag3.

In some cases, the cells are cultured in High IL-15/Low IL-2 (or High IL-15/Low IL-2, IL-7, IL-21 conditions) during activation (i.e., when the cells are being activated, for example by CD28/CD3 beads for transduction). In some cases, there are no exogenously added interleukins present during activation.

The manufacturing methods described herein can be used to expand T cell populations for a variety of therapeutic purposes. For example, the methods can be used to expand tumor infiltrating lymphocytes (TIL) isolated from a patient.

The manufacturing methods described herein can be used to expand a T cell population that is subsequently transfected with an RNA (e.g., an mRNA) encoding a T cell receptor (Krug et al. 2014 Cancer Immunology and Immunotherapy 63:999)

Patient-specific, autologous and allogeneic T cells (e.g., autologous or allogenic T_{CM/SCM/N} cells) can be engineered to express a chimeric antigen receptor (CAR) or T cell receptor (TCR) and the engineered cells can be expanded under High IL-15/Low IL-2 culture conditions or High IL-15/Low IL-2, IL-7, IL-21 conditions.

Described herein is a method for expanding T cells in culture medium that includes exogenously added IL-15 and little or no exogenously added IL-2 (and, optionally, little or no exogenously added IL-7 or IL-21). Also described is a method for activating a population of T cells that are cultured in culture media that includes exogenously added IL-15 and little or no exogenously added IL-2 (and, optionally, little or no exogenously added IL-7 or IL-21). Also described is a method for introducing a vector, e.g., a lentiviral or retroviral vector, expressing a T cell receptor (e.g., a CAR) into a population of T cells that have been activated and the expanding the cells in culture media that includes exogenously added IL-15 and little or no exogenously added IL-2 (and, optionally, little or no exogenously added IL-7 or IL-21). The introduction of the vector can take place in culture media that includes exogenously added IL-15 and little or no exogenously added IL-2 (and, optionally, little or no exogenously added IL-7 or IL-21) and includes components to cause T cell activation (e.g., CD3/CD28 beads). Alternatively, activation can take place in the absence of exogenously added interleukins.

Described herein is a method for preparing a population of human cells comprising T cells (i.e., CD3+ cells) optionally harboring a recombinant nucleic acid molecule encoding a T cell receptor, comprising: (a) providing a sample of human cells comprising T cells, wherein the T cells comprise: central memory T cells; memory stem T cells, and naïve T cells, wherein greater than 40% (greater than 45%, 50%, 55%, 60%, 65% or 70%) of the T cells are CD45RA+ and greater than 70% (greater than 75%, 80%, 85% or 90%) of the T cells are CD62L+; (b) activating the population of human cells comprising T cells; and (c) transducing or transfecting cells in the population of human cells comprising T cells with a recombinant nucleic acid molecule to provide a population of human cells comprising T cells harboring a recombinant nucleic acid molecule, wherein the method does not comprise a step of depleting cells expressing CD45RA, and then expanding the cells in culture media that includes exogenously added IL-15 and little or no exogenously added IL-2 (and, optionally, little or no exogenously added IL-7 or IL-21). In various embodiments: the recombinant nucleic acid molecule is a viral vector (e.g., a lentiviral vector or a retroviralvector encoding a T cell receptor such as a CAR); the method further comprises culturing the population of human cells comprising T cells harboring a recombinant nucleic acid molecule; the culturing step comprises the addition of exogenous IL-2 and exogenous IL-15 (and, optionally, little or no exogenously added IL-7 or IL-21); and the activating step comprises exposing the cells to an anti-CD3 antibody and an anti-CD28 antibody; and at least 80% (greater than 85%, 90%, 95%, or 98%) of the cells in the isolated population of cells comprising T cells are T cells. In some embodiments, step (c) is omitted and the cells are treated subsequent to expansion to introduce an RNA molecule encoding a T cell receptor such as a CAR. The RNA can be introduced into the expanded cells by electroporation or another suitable method and the transfected cells will transiently express the T cell receptor.

Described herein is method for preparing a population of human cells comprising T cells (i.e., cells that express CD3 or CD3+ cells), wherein the T cells comprise central memory T cells; memory stem T cells, and naïve T cells, wherein greater than 40% (greater than 45%, 50%, 55%, 60%, 65% or 70%) of the cells are CD45RA+ and greater than 70% (greater than 75%, 80%, 85% or 90%) are CD62L+, comprising: (a) providing an isolated population of human cells comprising T cells; (b) treating the isolated population of human cells comprising T cells to deplete cells expressing CD25 and cells expressing CD14 to prepare a depleted cell population; and (c) treating the depleted cell population to enrich for cells expressing CD62L, thereby preparing a population of human cells comprising T cells, wherein the T cells comprise central memory T cells; memory stem T cells, and naïve T cells, wherein greater than 40% of the cells are CD45RA+ (greater than 45%, 50%, 55%, 60%, 65% or 70%) and greater than 70% are CD62L+ (greater than 75%, 80%, 85% or 90%), wherein the method does not comprise a step of depleting cells expressing CD45RA, and then expanding the cells in culture media that includes exogenously added IL-15 and little or no exogenously added IL-2.

The population of T cells expanded in culture media that includes exogenously added IL-15 and little or no exogenously added IL-2 (and, optionally, little or no exogenously added IL-7 or IL-21) can be a population of human cells comprising T cells (i.e., cells that express CD3 or CD3+ cells), wherein the T cells comprise central memory T cells; memory stem T cells, and naïve T cells, wherein greater than 40% (greater than 45%, 50%, 55%, 60%, 65% or 70%) of the cells are CD45RA+ and greater than 70% (greater than 75%, 80%, 85% or 90%) are CD62L+, wherein the population is prepared by a method comprising: providing an isolated population of human cells comprising T cells (e.g. PBMC from a donor); treating the isolated population of human cells comprising T cells to deplete cells expressing CD25 and deplete cells expressing CD14 to prepare a depleted cell population; and treating the depleted cell population to enrich for cells expressing CD62L, thereby preparing a population of human cells comprising T cells, wherein the T cells comprise central memory T cells; memory stem T cells, and naïve T cells, wherein greater than 40% (greater than 45%, 50%, 55%, 60%, 65% or 70%) of the cells are CD45RA+ and greater than 70% (greater than 75%, 80%, 85% or 90%) are CD62L+, wherein the method does not comprise a step of depleting cells expressing CD45RA. In various embodiments: less than 15% (less than 12%, 10%, 8%, 6%) of the T cells in the population of human cells are CD14+ and less than 5% (less than 4%, 3% or 2%) of the T cells are CD25+; at least 40% (greater than 45%, 50%, 55%, 60%, 65% or 70%) of the T cells are CD4+ and CD62L+ or CD8+ and CD62L+; at least 10% (greater than 15%, 20%, 25%, 30%, 35%, or 40%) of the T cells are CD8+ and CD62L+; less than 60% (less than 55%, 50%, 45%, 40%, 35%, 30%, 24%, 20% or 15%) of the T cells are CD45RO+. The population of T cells can be primarily CD4+ cells (greater than 60, 70, 80 or 90% CD4+ cells) or primarily CD8+ cells (greater than 60, 70, 80 or 90% CD8+ cells).

Also described herein is a method of treating cancer, autoimmunity or infection comprising administering to a patient in need thereof a pharmaceutical composition comprising a human cell population manufactured under High IL-15/Low IL-2 culture conditions or High IL-15/Low IL-2, IL-7, IL-21 conditions described herein.

In some cases, the cells are autologous to the patient being treated and in some cases they are allogenic to the patient being treated.

### BRIEF DESCRIPTION OF THE DRAWINGS

FIG. 1: depicts certain marker expression data for various T cell subsets.

FIG. 2: schematically depicts the generation and culturing of a T cell populations.

FIG. 3: depicts the results of studies showing that CAR T cells expanded in the presence of IL-15 and in the absence of exogenously added IL-2 have improved in vivo antitumor activity. (A) Schematic representation of the experimental design to assess the antitumor effects of CD19 targeted CAR-T cells expanded with various cytokine combination. The CD19 CAR has been previously reported (Wang et al. 2015 Clinical Cancer Research 21:2993; Jonnalagadda et al. 2014 Molecular Therapy 23:757) and is comprised of the FMC63 scFv, a modified IgG4-Fc linker mutated, CD28 transmembrane domain and CD28-CD3ζ endodomains. The CAR cassette also includes a T2A ribosomal skip sequence followed by a truncated EGFRt for cell to detect transduced cells. (B) Bioluminescence imaging of tumor progression in mice engrafted with Raji tumor cells and treated with CAR T cells. (C) Kaplan Meier survival curve of mice after infusion of CART cells.

FIG. 4: depicts the results of studies showing that CAR T cells expanded in IL-15 in a long-term culture sustain their antitumor activity. CART cells were expanded in either IL-2 or IL-15 cytokine. At various time-points, cells were collected and assessed for their antitumor activity against Raji tumors in vivo. (A) Bioluminescence imaging (BLI) of tumor progression in mice engrafted with Raji tumor cells and treated with CART cells. (B) Photon flux of tumor cells with and without treatment acquired via BLI at various time-points (n=6). (C) Kaplan Meier survival curve of mice after infusion of CART cells.

FIG. 5: depicts the results of a study showing that IL-15 preserves less-differentiated memory phenotype of CAR T cells during ex vivo expansion. CAR T cells were expanded in either IL-2 or IL-15 cytokine. At various time-points, cells were collected and assessed for changes in memory phenotype. T cells were harvested on days 14 and 32 and flow cytometry analysis of their phenotype was conducted. (A) Pie chart shows reduction in frequency of CD45RA+CD62L+ cells cultured in IL-2 over time. (B) Flow cytometry analysis shows sustained CD27 expression in T cells cultured in IL-15

FIG. 6: depicts the results of a study showing that IL15 prevents expression of T cell exhaustion markers over long-term ex vivo culture. T cells cultured in either IL2 or IL15 cytokines were analyzed for exhaustion phenotypes on days 14, 23 and 32. Flow cytometry analysis shows over time increased expression of Lag3 (Top) and 2B4 (bottom) in T cells cultured in IL2. Graphs are summary data obtained from two different donors.

FIG. 7: A) Flow cytometric analysis of naïve/memory T cells cultured in different cytokine combination during ex vivo expansion (Day 18-20 after initiation of culture) showing frequency of marker expression as indicated on the labelled axes (left). Histogram plot showing CCR7 expression in total CD8 T cells (top right). Data shown are representative of two independent experiments. Bar graphs are gated on CD8+ cells, and summarize CD27+ (middle right) and CD45RO+ (bottom right) expression from two different donors. B) Flow cytometric analysis after 18-20 days of ex vivo expansion of indicated inhibitory molecules. Data shown are representative of two independent experiments. Bar graphs showing frequency of CD8+ T cells expressing 2B4 (top) and Lag3 (bottom). C) Schematic of in vivo CART cell therapy against Raji tumors in NSG mice to compare antitumor efficacy of CART cells cultured in different cytokine combinations. D) Bioluminescent images compares tumor progression over time in treated and untreated groups (n=6-8 mice per group). E) Kaplan Meier survival curve depicts overall survival of mice bearing Raji lymphoma untreated or treated with Mock or CD19 CART cells. p-value shown is IL-15compared to our standard cytokine condition (IL-2IL-15low). F) Comparison in expression of inhibitory receptors on CART cells harvested from animals 17 days post therapy. Each point is one animal, while mean is indicated by horizontal bars; *p<0.05; **p<0.01; ***p<0.001; ****p<0.0001 (two-tailed t test) is considered significant. Data is representative of two independent studies.

FIG. 8: Characterization of T cells product post enrichment process. Flow cytometry analysis of T cells post enrichment processes.

FIG. 9: IL-15 preserves the naïve/memory CAR T cell phenotype. A) Pie charts summarizing flow cytometric analysis of changes in CD45RA+CCR7+ expression over time in CART cells cultured in IL-21L-15low compared with IL-15. Data shown is representative of three independent donors. B) Frequency of CAR T cells with less differentiated phenotype (CD27+CD62L+ and CD127+CD62L+) in both culture conditions over time from three different donor cells. C) Quantitative RT-PCR analysis of key genes involved in naïve/memory T cells formation in CD4+ and CD8+ IL-15-cultured CAR T cells. Results are presented relative to beta-actin gene. D) Heat map depicts global changes in genes regulating T cell differentiation in CD8+ T cells between days 14 to 32. Red and white indicates high and low expression, respectively. E) Changes in frequency of CD45RA+ and CD45RO+ T cells over time shows in IL-15-cultured CAR T cells. Data are presented as mean±SEM and *p<0.05; **p<0.01; ***p<0.001; ****p<0.0001 (two-tailed t test) of two independent studies.

FIG. 10: T cells cultured in IL-15 have reduced effector phenotype. A) Effector function was measured by frequency of CD107a+ and IFNγ+ after co-culturing CART cells with target cells (CD19+; Raji,) at a 1:1 Effector:Target ratio for 5 hours. B) Quantitative RT-PCR expression of indicated effector genes in CD4+ and CD8+ T cells. C) Robust multichip analysis (RPKM)-normalized intensity of selected genes progressively down or up-regulated in CD8+ T cells.

FIG. 11: IL15-cultured T cells represent a distinct, less-differentiated T cell memory subset. A) Heat map of differentially expressed genes (P<0.01, false discovery rate <5%, Benjamini-Hochberg's method) among CD8+ T cell subsets. Red and green colors indicate increased and decreased expression, respectively. B) MDS analysis of differentially expressed genes (P<0.01, false discovery rate <5%). Numbers represent the differentially regulated genes among each CD8+ T cell subset (P<0.01 (t test) and > twofold change in expression). C) Robust multichip analysis (RPKM)-normalized intensity of selected genes progressively down or up-regulated in CD4+ subset at early time-point (Day 14).

FIG. 12: IL-15 promotes T cell survival and inhibits up-regulation of inhibitory receptors associated with T cell exhaustion. A) Intracellular levels of active capsase-3 was measured by flow cytometry. Bar graph displays percent CD3+

Capsase-3+ population from three different donors (right), and one representative density plot of intracellular caspase-3 staining on day 32 (left). B) Western Blot analysis shows level of anti-apoptotic protein Bcl2 in T cells cultured in IL-21L-15low or IL-15 over time. C) Flow cytomeric analysis shows frequency of T cells positive for inhibitory receptors such as lag3 (top) and 2B4 (bottom). Flow cytometry plots over time from one representative donor is shown (left), and bar graphs are presented as mean±SEM from three independent donors. *p<0.05; **p<0.01; ***p<0.001 (two-tailed t test, D) Robust multichip analysis (RPKM)-normalized intensity of selected genes progressively down or up-regulated in T c ells (CD8+) cultured in IL-21L-15low or IL-15.

FIG. 13: IL-15 reduces mTOR activity and glycolysis. A) Immunoblot analysis of Glut1, CPT1a, p-rps6 and pAkt proteins in T cells cultured in IL-2IL-15low or IL-15. GAPDH was used as a loading control. B) Quantitative RT-PCR analysis of Glut1 (slc2a1) and CPT1a expressions in CD4 and CD8 T cells. Results are presented relative to actin gene. C) Heat map of RNA-sequencing analysis of sorted CD8+ T cell subsets, highlighting changes in the canonical genes associated with glycolysis, fatty acid oxidation (FAO) between days 14 and 32. Red indicates greatest increase and white indicates no change between day 14 and day 32 expression.

FIG. 14: IL15-mediated reduced mTOR activity results in more stem-like population. A) Flow cytometry analysis shows changes in CD45RA+CCR7+CD3+ T cells cultured in IL-21L-15low, IL-15 or IL-21L-15low+rapamycin (100 nM (left), summarized in a pie chart (right). B) Bar graph shows changes in the frequency of CD62L+CD27+and CD62L+CD27+ T cells cultured in above conditions. Data presented are mean ±SEM of two experiments. C) Immunoblot analysis of phosphorylated rps6 protein confirms reduction of mTORC1 activity in T cells cultured in IL-15 and IL-21L-15low+Rapa. GAPDH was used as a loading control. Data is representative two independent studies.

FIG. 15: T cells cultured in the presence of IL-15 exhibit enhanced self-renewal capacity and maintain antitumor activity in vitro. CAR T cells were co-cultured with tumor cells (CD19+; Raji) at a 1:2 Effector:Target ratio for 7 days. After 7 days, number of A) CART cells and B) tumor cells were counted by flow cytometry and graphed.(Data are presented as mean±SEM and *p<0.05; **p<0.01; ***p<0.001; ****p<0.0001 (two-tailed t test) of three independent studies.

FIG. 16: T cells cultured with IL-15 have superior antitumor activity and remain detectable post adoptive T cell transfer. Mice bearing Raji lymphoma were untreated or treated with 1×106 mock or CD19 CART cells three days after tumor engraftment. T cells were thawed and injected after cryopreservation at the indicated number of days in ex vivo culture. A) Bioluminescent images compares tumor progression 19 days after adoptive transfer of T cells maintained in the indicated cytokine conditions (n=6-8 mice per group). B) Bioluminescent flux plot quantifying tumor burden in response to different treatment groups over time. Data is shown as mean±SEM. C) Kaplan Meier survival curve depicts overall survival. D) Frequency of circulating CAR T cells 10 days post CAR T cell therapy identified by flow cytometry using antibodies to human CD3 and human CD45 (left). Data are presented as mean±SEM of 6-8 individual animals and *p<0.05; **p<0.01; ***p<0.001; ****p<0.0001 (two-tailed t test) of two independent studies (right).

### DETAILED DESCRIPTION

The T cell compartment includes T cell subsets that are at different stages of differentiation. These subsets arise from differentiation of Naïve T cells (TN), which are CD45RA+, CD62L+, CD28+, and CD95−. Among the stem cell-like subsets are Memory Stem Cells (T_{SCM}), which are CD45RA+, CD62L+, CD28+, and CD95+. These cells differentiate into Central Memory Cells (T_{CM}), which are CD45RO+, CD62L+, CD28+, and CD95+. T_{CM} differentiate in Effector Memory Cells (T_{EM}), which are CD45RO+, CD62L−, CD28+/−, and CD95+. The T_{EM} differentiate to Effector T cells (T_{E}) which are CD45RO+, CD62L+, CD28+, and CD95+.

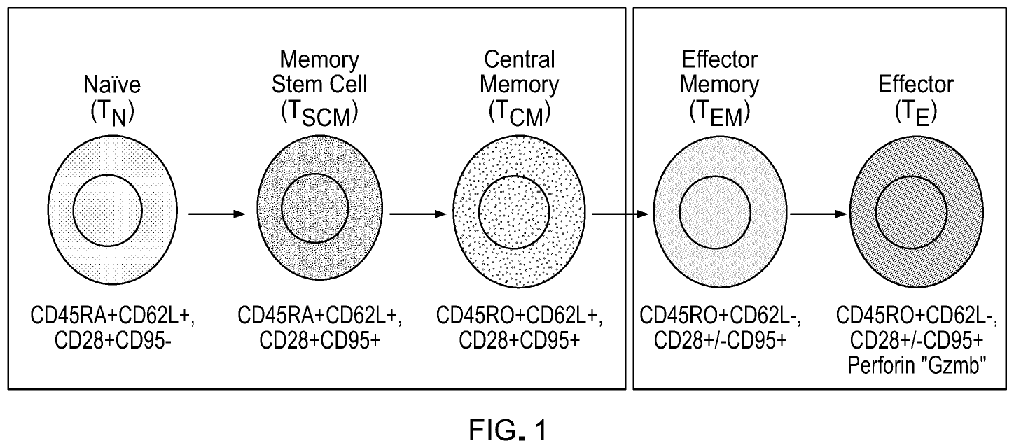

Memory Stem T Cells (T_{SCM}) are present at a low level in the T cell compartment, but appear to have significant self-renewal and proliferative potential. While they resemble naïve T cells (T_{N}) in that they express CD45RA+ and CD62L+, they can be distinguished from T_{N} by their expression of CD95 (FIG. 1). T_{SCM} can be generated from T_{N} by stimulation with CD3/CD28 beads in the presence of IL-7 and IL-15. They also can be expanded in the presence of Wnt/β-catenin pathway activation (Cieri et al. 2013 Blood 121:573; Gattinoni et al. 2009 Nature Medicine 15:808).

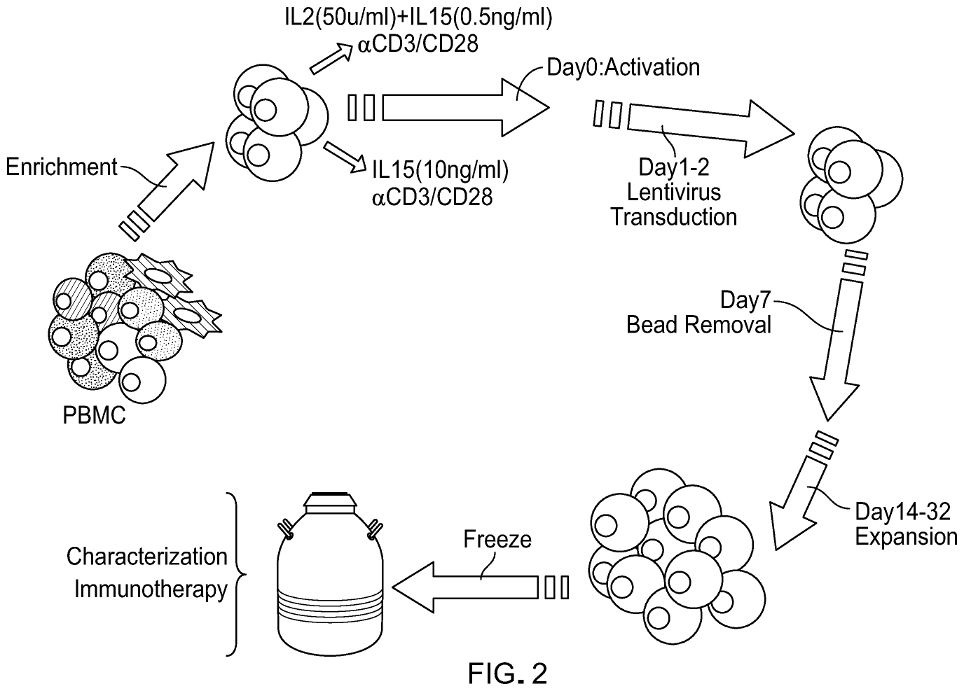

Central Memory T Cells (T_{CM}), which are more abundant in PBMC than are T_{SCM}, are a well-defined memory T cell subset with high self-renewal and proliferative potential. There is evidence that T_{CM} persist following adoptive transfer better than Effector T cells (T_{E}) (Berger et al. 2008 Journal of Cellular Immunology 118:4817; Wang et al 2011 Blood 117:1888). T_{CM} can be enriched from PBMC for T cell therapy manufacturing based on their CD45RA−CD45RO+CD62L+ phenotype (FIG. 2) (Wang et al. 2012 J Immunotherapy 5:689). There is some evidence that T_{CM} behave as adult stem cells. Studies in mice demonstrated that: single cell transfer of T_{CM} over three generations demonstrated that T_{CM} can provide full immune reconstitution; that T_{CM} expand to produce more T_{CM}; and that T_{CM} differentiate to T_{EM}/T_{E} (Graef et al. 2014 Immunity 41:116; Gattioni et al. 2014 Immunity 41:7).

The various T cell populations described can be genetically engineered to express, for example, a CAR or a T cell receptor. A CAR is a recombinant biomolecule that contains an extracellular recognition domain, a transmembrane region, and one or more intracellular signaling domain. The term “antigen,” therefore, is not limited to molecules that bind antibodies, but to any molecule that can bind specifically to any receptor. “Antigen” thus refers to the recognition domain of the CAR. The extracellular recognition domain (also referred to as the extracellular domain or simply by the recognition element which it contains) comprises a recognition element that specifically binds to a molecule present on the cell surface of a target cell. The transmembrane region anchors the CAR in the membrane. The intracellular signaling domain comprises the signaling domain from the zeta chain of the human CD3 complex and optionally comprises one or more co-stimulatory signaling domains. CARs can both to bind antigen and transduce T cell activation, independent of MHC restriction. Thus, CARs are “universal” immunoreceptors which can treat a population of patients with antigen-positive tumors irrespective of their HLA genotype. Adoptive immunotherapy using T lymphocytes that express a tumor-specific CAR can be a powerful therapeutic strategy for the treatment of cancer.

The CAR can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region can be inserted into an expression vector and used to transform a suitable expression host cell line, preferably a T lymphocyte cell line, and most preferably an autologous T lymphocyte cell line. Alternatively, the coding region can be transiently expressed by an RNA that is introduced into the T cells after expansion using the methods described herein.

Various CAR suitable for expression by T_{CM/SCM/N} cells include, for example, those described in: WO 2016/044811; WO 2104/144622; WO 2002/077029; and WO/US2014/0288961.

### Example 1

### Preparation of T_{CM/SCM/N} Cells

A variety of methods can be used to produce a population of human T_{CM/SCM/N} cells. For example, a population of T_{CM/SCM/N} cells can be prepared from a mixed population T lymphocytes. The population of T lymphocytes can be allogenic to or autologous to the subject ultimately treated using the cells and can be obtained from a subject by leukopheresis or blood draw.

The following method is an example of one that can be used to obtain a population of T_{CM/SCM/N} cells from T lymphocytes obtained by leukapheresis or other means. Peripheral blood is collected by leukapheresis or peripheral blood draw. Day 1 of a typical manufacturing cycle is the day the ficoll procedure takes place. The subject's leukapheresis product is diluted with EDTA/PBS and the product is centrifuged at 1200 RPM for 10 minutes at room temperature with maximum brake. After centrifugation, the platelet-rich supernatant is removed and the cell pellet is gently vortexed. EDTA/PBS is used to re-suspend the vortexed cell pellets in each conical tube. Each tube is then underlayed with ficoll and centrifuged at 2000 RPM for 20 minutes with no brake at room temperature. Following centrifugation, the PBMC layer from each tube is transferred into another conical tube. The cells are centrifuged at 1800 RPM for 15 minutes with maximum brake at 4° C.

After centrifugation, the cell-free supernatant is discarded and the cell pellet is gently vortexed. The cells are washed twice using EDTA/PBS each time, and a third time using PBS. Cells are centrifuged each time at 1200 RPM for 10 minutes with maximum brake at 4° C. After the final PBS wash, the vortexed cell pellet is resuspended in complete X-VIVO 15 media (X-VIVO™ media with 10% FBS) and transferred to a transfer bag. The bag with washed PBMC is kept overnight on a rotator at room temperature on the bench top for immunomagnetic selection the next day.

Next, selection procedures are used to both to deplete the cell population of cells expressing certain markers and to enrich the cell population for cells expressing certain other markers. These selection steps preferably occur on day two of the manufacturing cycle. The cell population is substantially depleted for cells expressing CD25 and CD14. Importantly, the cell population is not substantially depleted for cells expressing CD45RA. Briefly, cells resuspended in labeling buffer (LB; EDTA/PBS with 0.5% HSA), and incubated with anti-CD14 and anti-CD25 Miltenyi antibodies for CliniMACS® depletion, and the composition is gently mixed and then incubated for 30 minutes on a rotator at room temperature on the bench top.

The depletion step is performed on a CliniMACS® device using a depletion tubing set. The recovered cells following the depletion step are transferred into tubes and centrifuged at 1400 RPM for 15 minutes with maximum brake at 4° C.

The cell-free supernatant is removed and the cell pellet is gently vortexed and resuspended. To enrich for cells expressing CD62L, the cell suspension is treated with anti-CD62L-biotin (made at the City of Hope Center for Biomedicine and Genetics), gently mixed and incubated for 30 minutes on a rotator at room temperature on the bench top.

Following the incubation period, LB is added to the tube and cells are centrifuged at 1400 RPM for 15 minutes at maximum brake at 4° C. The cell-free supernatant is removed and the cell pellet is gently vortexed. LB is added to resuspend the cell pellet in the tube and the resuspended cells are transferred to a new transfer bag. Anti-biotin (Miltenyi Biotec) reagent is added and the mixture is gently mixed and incubated for 30 minutes on a rotator at room temperature on the bench top.

The CD62L enrichment step is performed on a CliniMACS® device using a tubing set. The product of this enrichment can be frozen for storage and later thawed and activated

To provide an intermediate holding step in the manufacturing, the option exists to freeze cells following the selection process. The cells are pelleted by centrifugation at 1400 RPM for 15 minutes with max break at 4° C. The cells are resuspended in Cryostor® and aliquoted into cryovials. The vials are transferred to a controlled cooling device that can cool at about 1° C./minute (e.g., a Nalgene® Mr. Frosty; Sigma-Aldrich) the cooling device is immediately transferred to a −80° C. freezer. After three days in the −80° C. freezer, the cells are transferred into a GMP LN2 freezer for storage.

We have found that cryopreserved cells exhibit good recovery and viability, maintain the appropriate cell surface phenotype when thawed up to 8.5 months after cryopreservation, and can be successfully transduced and expanded in vitro upon thawing.

Alternatively, freshly enriched T_{CM/SCM/N} cells can be activated, transduced and expanded as described below.

### Example 2

### Activation, Lentiviral Transfection and Culturing in the Presence of Certain Cytokines

Human T cells, either bulk PBMC or enriched T cell subsets, are stimulated as for example with GMP Dynabeads® Human T expander CD3/CD28 (Invitrogen) at a 1:3 ratio (T cell:bead). On day 0 to 3 of cell stimulation, T cells are transduced, for example with a CAR-expressing lentivirus, in X Vivol5 containing 10% fetal calf serum (FCS) with 5μg/mL protamine sulfate (APP Pharmaceutical), and with exogenously added cytokines (i.e., final concentration 10 ng/mL rhIL-15). The next day following lentivirus transduction, media is exchanged or cultures diluted 1:2 to in X Vivo 15 containing 10% FCS and cytokines. Cultures are then maintained at 37° C., 5% CO₂ with addition of X-Vivo15 10% FCS as required to keep cell density between 3×10⁵ and 2×10⁶ viable cells/mL, with cytokine supplementation (i.e, final concentration of 10 ng/mL rhIL-15) every Monday, Wednesday and Friday of culture. On day 7 to 10 following T cell stimulation, the CD3/CD28 Dynabeads are removed from cultures using the DynaMag-50 magnet (Invitrogen). Cultures are propagated until day 8 to 32 days and then cryopreserved. Over the duration of the culture, cells are supplemented with a combination of cytokines [IL2 (50 U/mL)+IL15 (0.5 ng/mL), IL7 (10 ng/mL)+IL15 (10 ng/mL) or IL7 (10 ng/mL)+IL15 (10 ng/mL)+IL21 (10 ng/mL), or IL-15 only (10 ng/mL). Two thirds of the culture media is removed and fresh media consisting of above cytokine combination is added at a 0.6×10⁶ cells/mL concentration. Exogenous cytokine addition is optional during the CD3/CD28 bead stimulation phase, however, it is essential during the expansion phase following removal of the beads. The amount of cytokine added to reach a desired level of exogenously added cytokine is based in the assumption that any media not replaced when fresh media is added is essentially free of any previously exogenously added cytokine.

### Example 3

### CAR T Cells Expanded in the Presence of IL-15 and in the Absence of Exogenously added IL-2 have Improved In Vivo Antitumor Activity

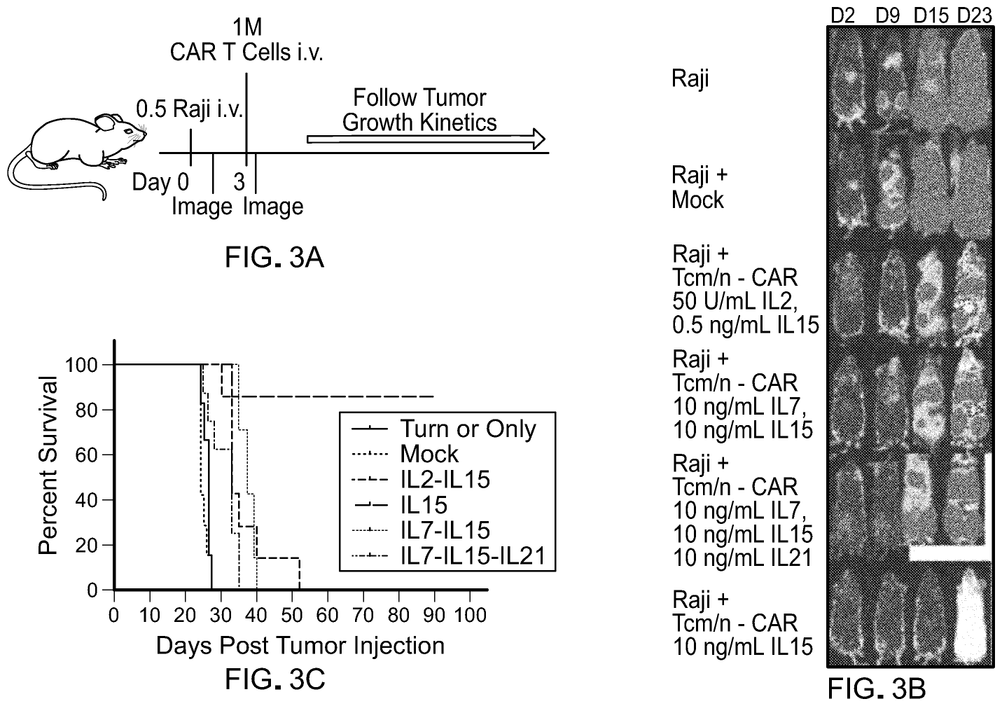

T_{CM/SCM/N} cells prepared and transduced as described above to express a CAR targeted to CD19 were expanded in the presence of 50 U/ml of IL-2 and 0.5 ng/ml of IL-15; 10 ng/ml of each of IL-7 and IL-15; 10 ng/ml of each of IL-7, IL-15 and IL-21 or 10 ng/ml of IL-15 only. The cells were injected into mice engrafted with Raji tumor cells. The experimental design is shown schematically in FIG. 3(A). Bioluminescence imaging of tumor progression in mice engrafted with Raji tumor cells and treated with CAR T cells is shown in FIG. 3(B) and Kaplan Meier survival curve of mice after infusion of CAR T cells is shown in FIG. 3(C).

As can be seen, in all conditions except IL-15 only, there was less than 50% survival by day 40. Importantly, excluding IL-2 and excluding IL-7 when IL-15 was present, improved anti-tumor activity.

### Example 4

### CAR T Cells Expanded Long Term in the Presence of IL-15 and in the Absence of Exogenously added IL-2 Sustain In Vivo Antitumor Activity

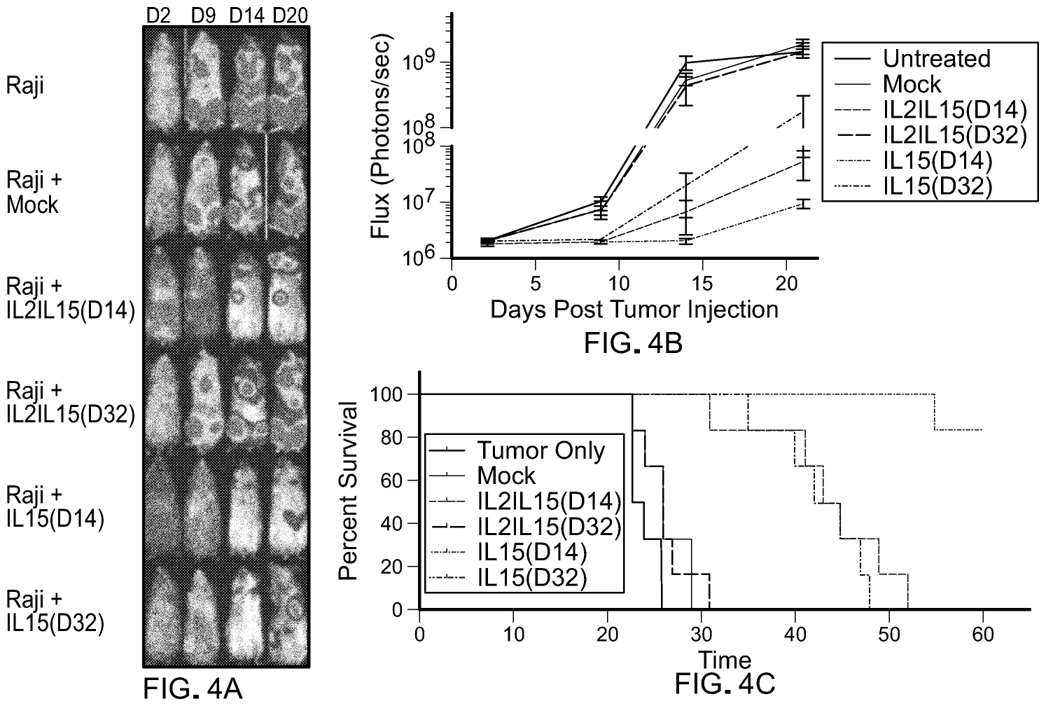

As shown in FIG. 4 (A)-(C), CART cells expanded in IL-15 in a long-term culture sustain their antitumor activity. CAR T cells were expanded in either IL-2 (50 U/ml) with low IL-15 (0.5 ng/mL) or IL-15 only (10 ng/ml). At various time-points, cells were collected and assessed for their antitumor activity against Raji tumors in vivo. FIG. 4(A) depicts bioluminescence imaging of tumor progression in mice engrafted with Raji tumor cells and treated with CAR T cells. FIG. 4(B) present photon flux of tumor cells with and without treatment acquired via bioluminescence imaging at various time-points (n=6) and FIG. 4(C) presents Kaplan Meier survival curve of mice after infusion of CAR T cells.

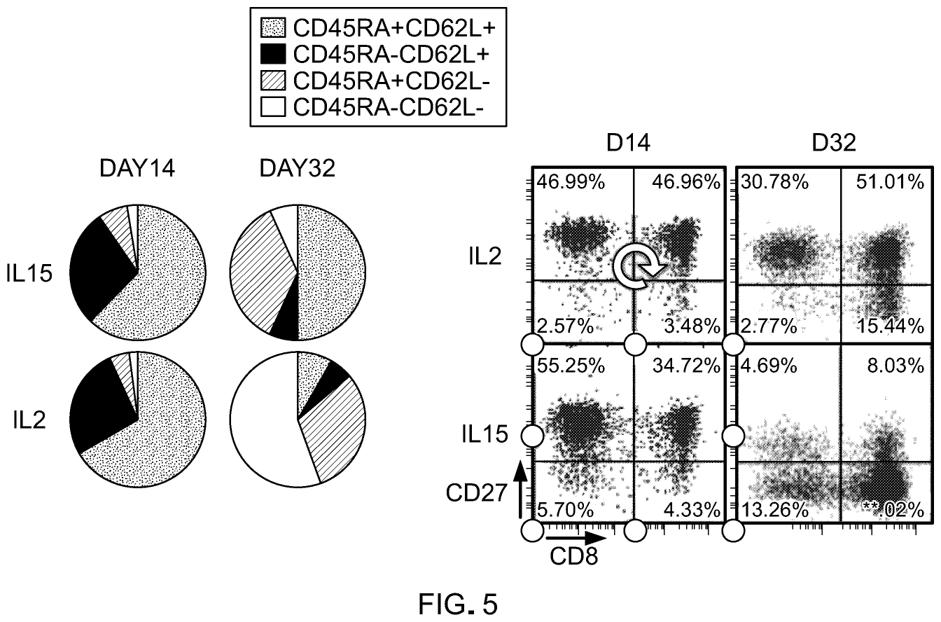

FIG. 5 depicts the results of a study showing that IL-15 preserves less-differentiated memory phenotype of CAR T cells during ex vivo expansion. CAR T cells were expanded in either IL-2 at 50 U/ml with low IL-15 (0.5 ng/mL) or IL-15 at 10 ng/ml. At various time-points, cells were collected and assessed for changes in memory phenotype. T cells were harvested on days 14 and 32 and flow cytometry analysis of their phenotype was conducted. (A) Pie chart shows reduction in frequency of CD45RA+CD62L+ cells cultured in IL-2 over time. (B) Flow cytometry analysis shows sustained CD27 expression in T cells cultured in IL-15.

As can be seen when cells were expanded in the presence of IL-2 at 50 U/ml with low IL-15 (0.5 ng/mL) for 14 days, 50% survival was between 45 and 50 days, but this decreased to between 25 and 30 days when the cells were expanded for 32 days. In contrast, for cells expanded in IL-15 only at 10 ng/ml, 50% survival was between 45 and 50 days even when the cells had been expanded for 14 days and was far longer when the cells were expanded for 14 days.

### Example 5

### Expansion in the Presence of IL-15 Preserves Less-Differentiated Memory Phenotype of CAR T Cells Compared to Expansion in the Presence of IL-2

CAR T cells were expanded in either IL-2 (50 U/ml) with low IL-15 (0.5 ng/ml) or IL-15 (10 ng/ml). At various time-points, cells were collected and assessed for changes in memory phenotype. T cells were harvested on days 14 and 32 and flow cytometry analysis of their phenotype was conducted. FIG. 5(A) is shows reduction in frequency of CD45RA+CD62L+ cells cultured in IL-2 with low IL-15 over time. Cells culture in the presence of IL-15 only showed a higher proportion of CD45RA+CD62L+ cells. Flow cytometry analysis showed sustained CD27 expression in T cells cultured in IL-15 (FIG. 5(B)). Together this data indicates that IL-15 preserves the memory stem cell phenotype. Less differentiated T cell product has longer persistence and potentially enhanced self-renewal.

### Example 6

### Expansion in the Presence of IL-15 Reduces Expression of Exhaustion Markers During Long Term Ex Vivo Culture Compared to Expansion in the Presence of IL-2

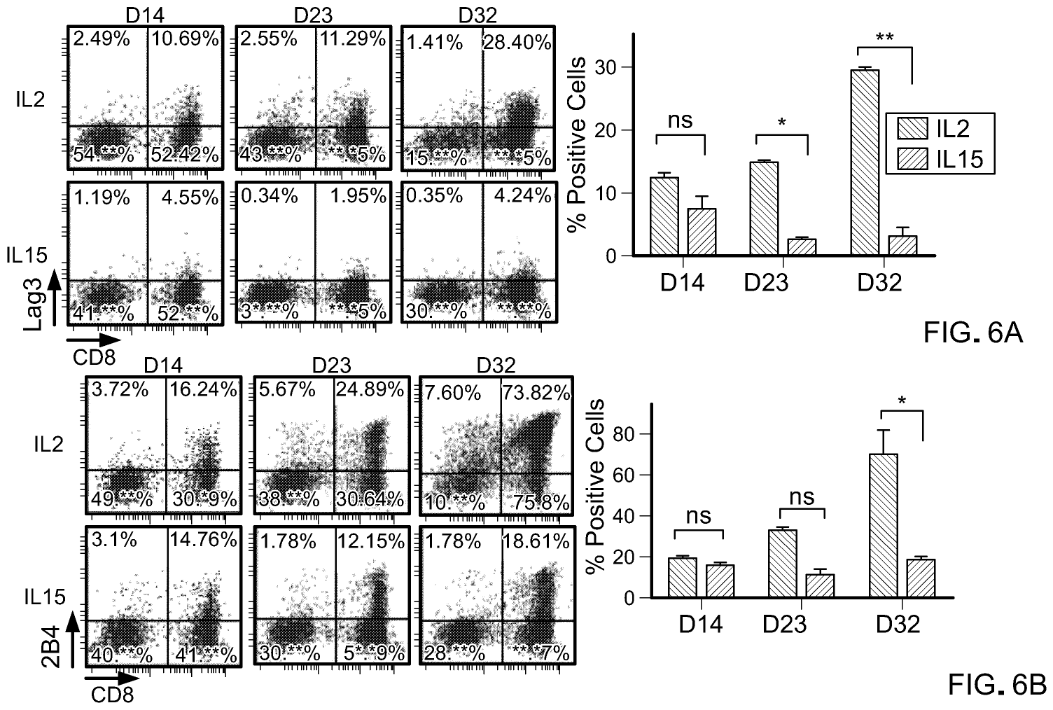

T cells cultured in either 50 U/ml of IL-2 with low IL-15 (0.5 ng/ml) or 10 ng/ml of IL-15 were analyzed for exhaustion phenotypes on days 14, 23 and 32. Flow cytometry analysis shows over time increased expression of Lag3 (FIGS. 6(A)) and 2B4 (FIG. 6(B)) in T cells cultured in IL-2. Exhaustion is a major defect in limiting T cell function. T cells with exhausted phenotype have impaired proliferation, persistence, and antitumor efficacy after adoptive transfer.

### Example 7

### CAR T cells Generated in Presence of IL-15 Exhibit Improved Antitumor Properties

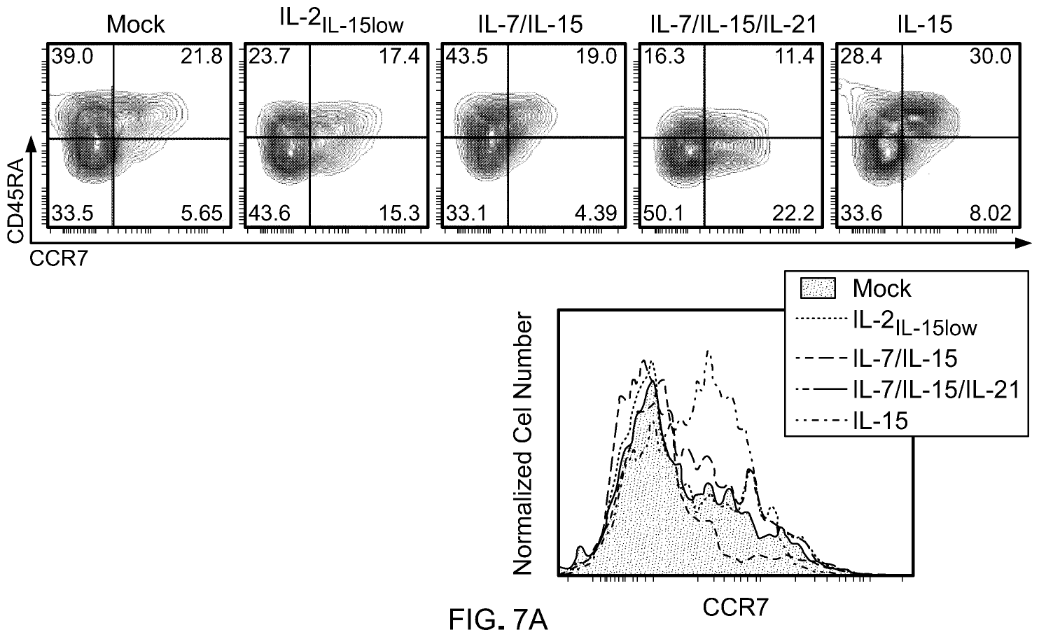

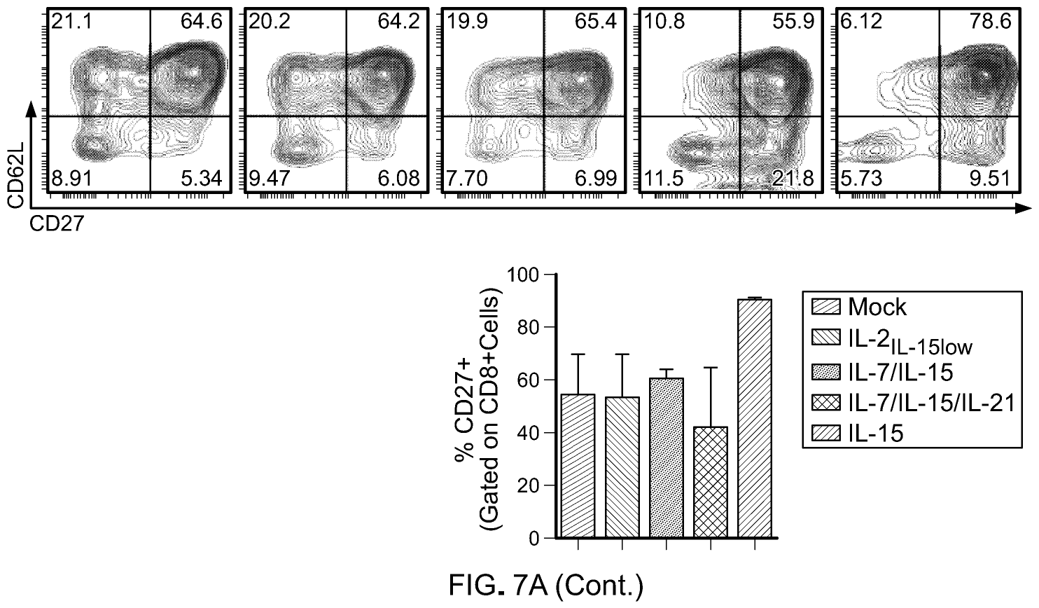

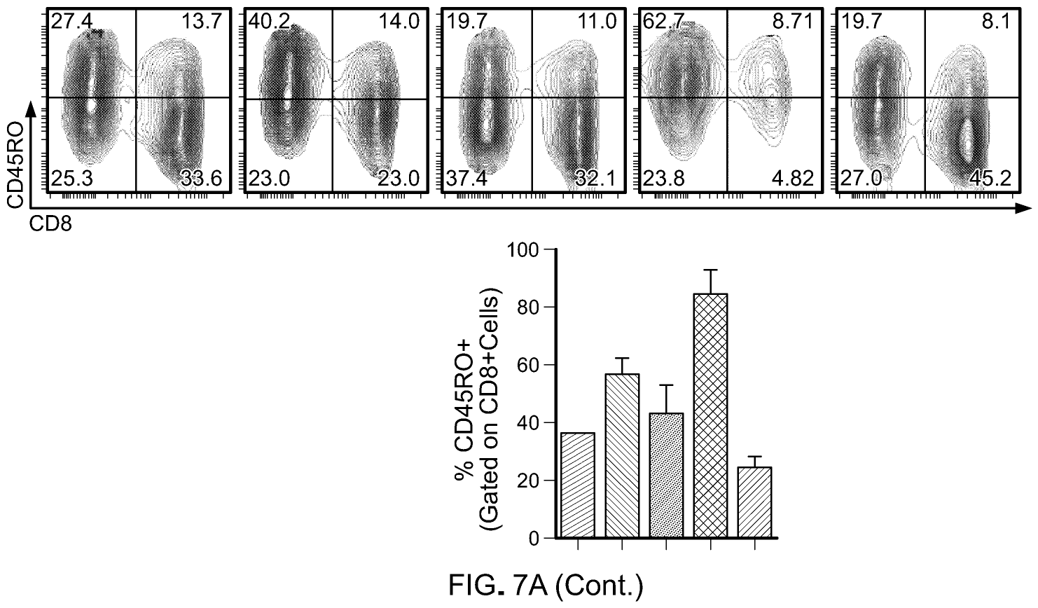

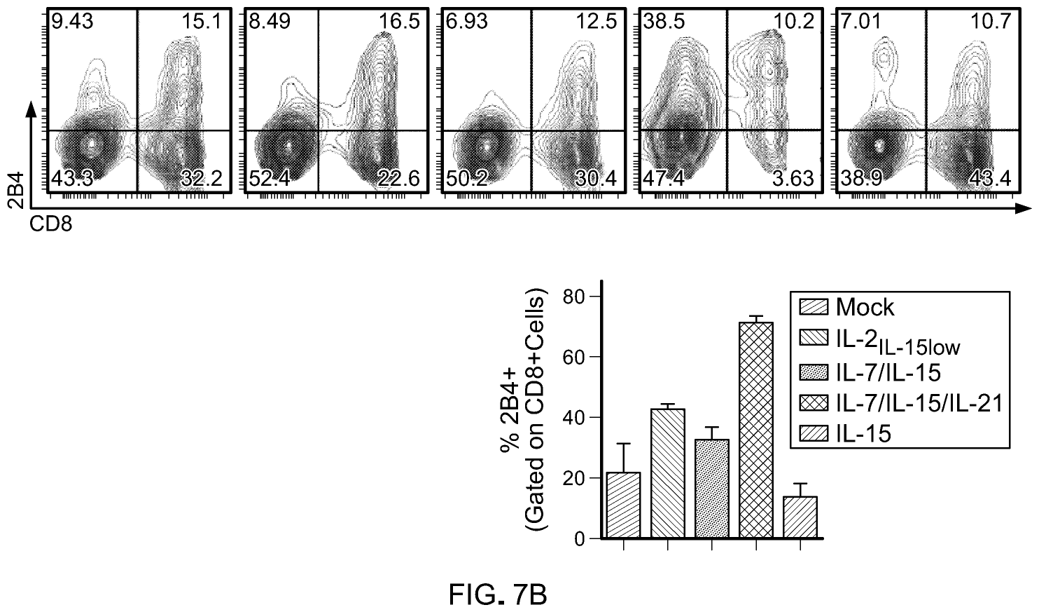

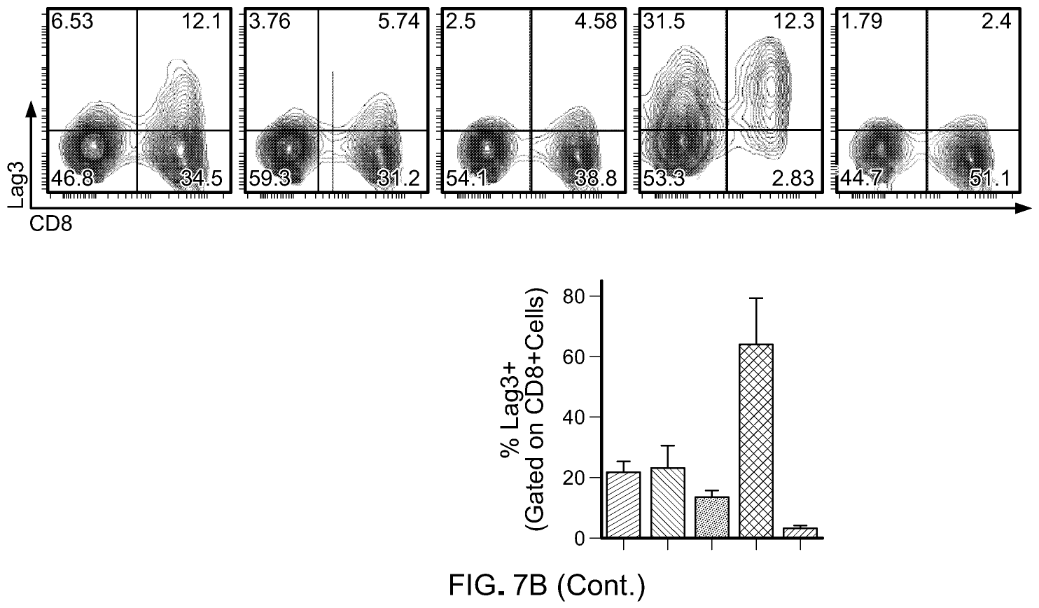

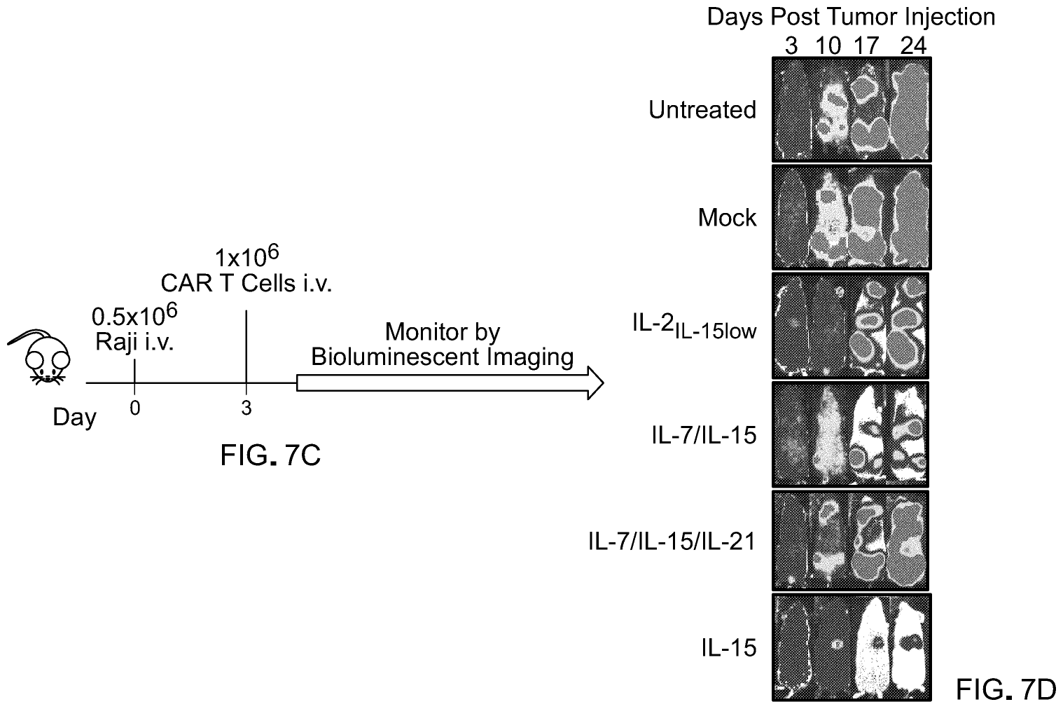

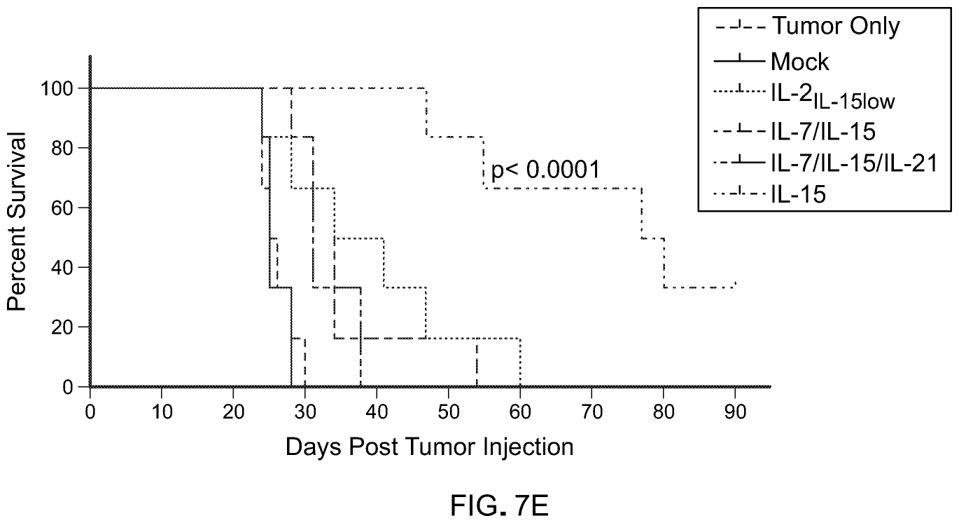

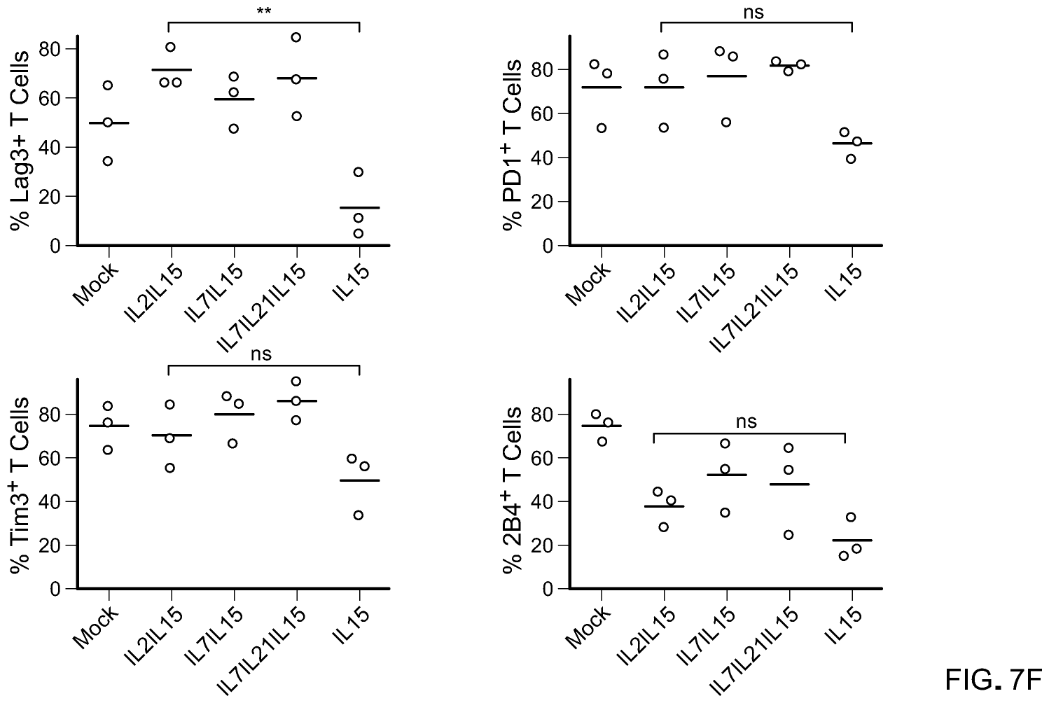

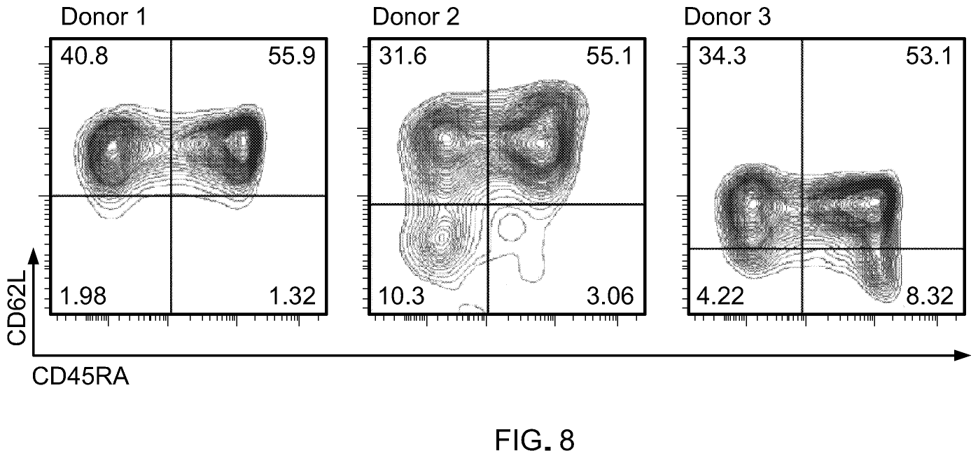

For this study, CD14⁺ and CD25⁺ cells were depleted from total PBMC product. CD62L⁺ positive T cells (total CD4 and CD8) were further positively selected. The product post-enrichment process contains 55±10% CD3⁺CD45RA⁺CD62L⁺ (FIG. 8) and 35±10% CD3⁺CD45RO⁺CD62L⁺ cells (data not shown) and therefore defined as naïve/memory T cells. Phenotypical analysis performed 18-20 days after the initiation of the culture indicated that 50-70% of the cells exhibited a CD45RA⁺CD62L⁺ phenotype (data not shown) with 30-55% expressing CD45RO (FIG. 7(A)) when culture with IL-2_{IL-15low}, IL-7/IL-15 or IL-15. In the presence of IL-15 alone, cells additionally expressed a more prominent CD45RA⁺CCR7⁺ and CD62L⁺CD27⁺ phenotype compared to the other culture conditions (FIG. 7(A)). Culture in IL-15 also prevented the up-regulation of inhibitory receptors such as 2B4 and Lag3 FIG. 7(B)) with no significant changes in PD-1 (data not shown). The observed changes were predominately detected in CD8⁺ T cells. Of note, T cells cultured in IL-7/IL-15/IL-21 exhibited increased expression of inhibitory molecules and only 7±5% CD45RA⁺CCR7⁺ T cells were preserved (FIG. 7(A) and FIG. 7(B)). Importantly, in vivo assessment of CART cell products showed superior antitumor activity of IL-15-cultured T cells against Raji cells, an aggressive CD19⁺ lymphoma mouse model. Furthermore, bioluminescence imaging indicated that CAR T lymphocytes generated in presence of IL-15 alone were endowed with significantly more potent antitumor activity compared to CAR T cells generated in the other cytokine conditions (FIG. 7(C), FIG. 7(D) and FIG. 7(E)). CAR T lymphocytes isolated from blood 17 days post CART cell therapy showed reduced expression of inhibitory molecules in the group treated with IL-15 CART cells (FIG. 7(F)). Together this data prompted further investigation on the effect of IL-15 on T cells as compared with our standard IL-2_{IL-15low} culture condition.

### Example 8

### CAR T Lymphocytes Generated with IL-15 Retain Features of Less Differentiated Cells

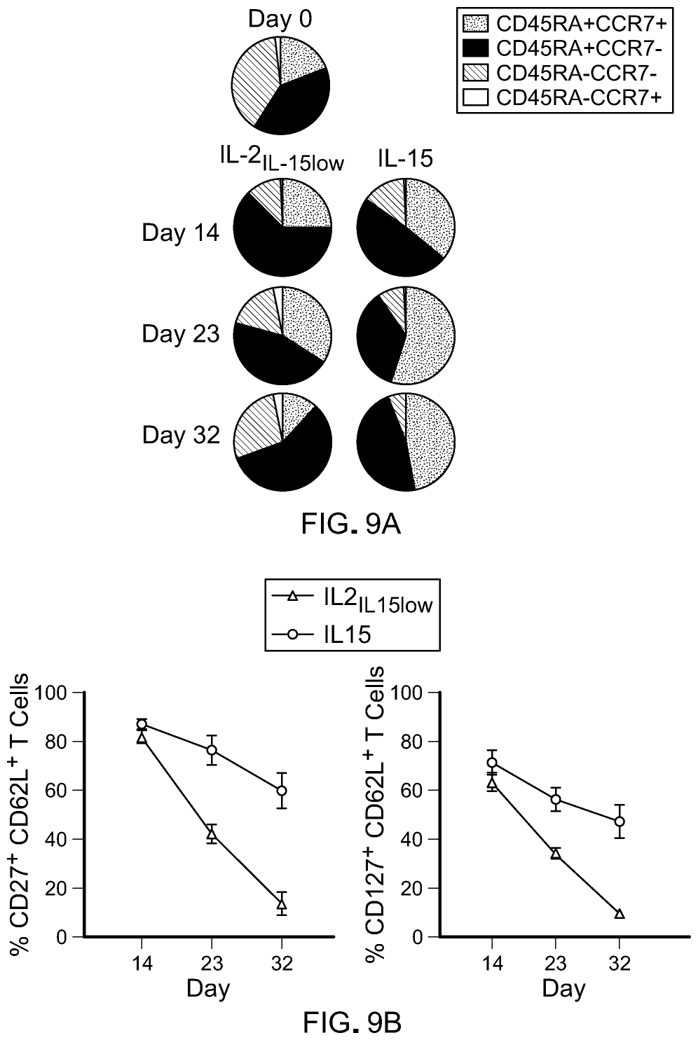

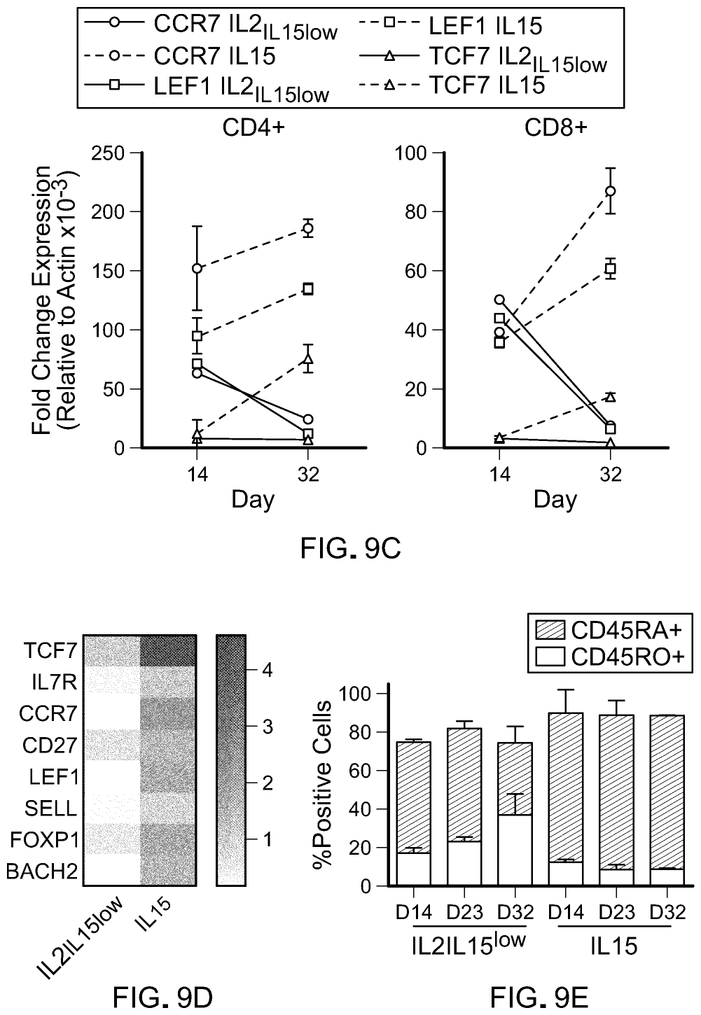

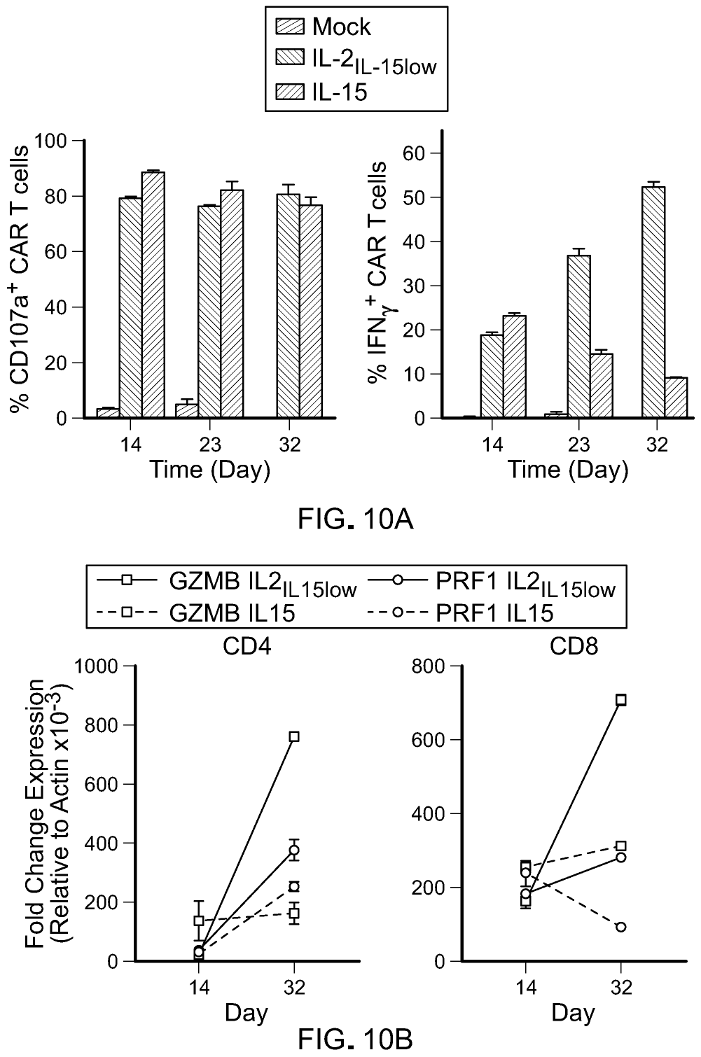

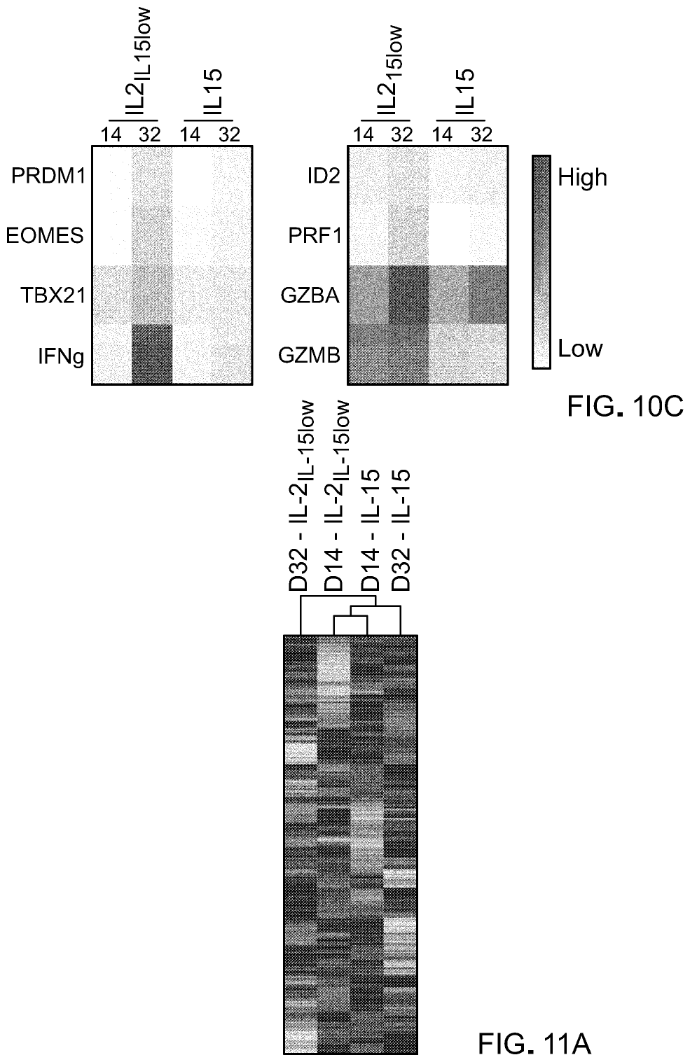

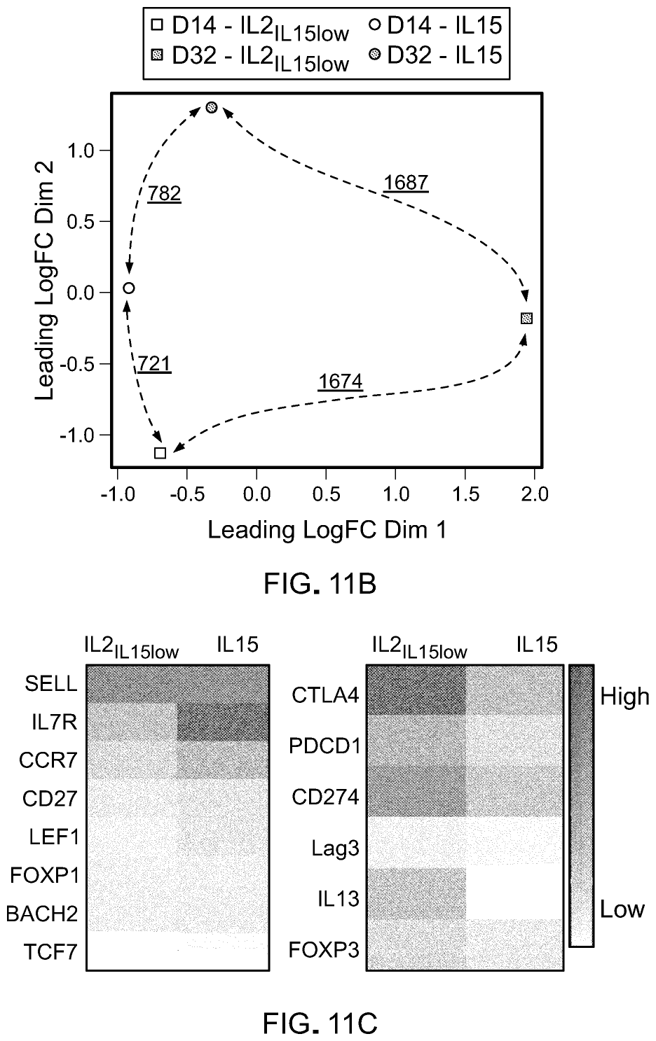

Previous studies have indicated that IL-2 promotes the generation of highly differentiated T cell subsets such as Tem and Teff. To assess whether the replacement of IL-2 by IL-15 may overcome this pitfall, we expanded CART cells over an extended period of time in the presence of IL-15 alone. Our data indicate that the IL-15 culture condition maintains a higher proportion of generated CAR T cells (both CD4+ and CD8+) that exhibit a naïve/memory phenotype (CD62L+CD45RA+CD45RO−) with increased expression of CCR7, CD27 and CD127 compared to CART lymphocytes generated with IL-21L-15low (FIG. 9(A), FIG. 9(B) and FIG. 9(E)). Further qPCR analysis and RNA sequencing studies confirmed up-regulation of key naive and memory-associated factors in IL-15-cultured cells compared with IL-2IL-15low cells (FIG. 9(C) and FIG. 9(E) and FIG. 11(C)). Consistent with these results, IL-15-generated CAR T cells expressed lower levels of IFNγ (marker of Teff cells) following antigen stimulation compared to CART lymphocytes generated with IL-2IL-15low (FIG. 10(A)). In line with this finding, both qPCR and RNA sequencing analysis revealed overall reduced expression of genes associated with effector phenotype in IL-15-cultured CART cells compared with IL-2IL-15low (FIG. 9(B) and FIG. 9(C)).

To confirm the phenotypic changes and to further globally assess the influence of IL-15 and IL-21L-15low on CART cell subsets, T cells expanded in IL-15 or IL-2IL-15low were sorted for CD8 and CD4 CAR+ T cells at different time points and gene-expression analyses were performed. Hierarchical clustering highlighted extensive differences in both CD8 and CD4 population among the two culture conditions. Multidimensional scaling (MDS) analysis showed that the IL-21L-15low and the IL-15 cultured cells exhibited different expression profiles by day 14 in culture (721 differentially expressed genes, P<0.01 and greater than twofold change in expression, FIG. 11(B)). Interestingly, by 32 days in culture, the IL-2IL-15low cultured cells had a drastically different gene expression profile compared to both the earlier IL-2IL-15low timepoint and the IL-15 cells (1674 and 1687, respectively), while the IL-15 cells clustered much closer to its earlier timepoint (782 differentially expressed genes).Furthermore, 123 genes were differentially expressed among the CD4+ T cell subsets (P<0.01 and greater than 1.5 fold change in expression) at the early time-point (data not shown). These data thus confirm that CART lymphocytes generated with IL-15 exhibit characteristics of less differentiated cells compared to their counterparts generated with IL-21L-15low.

### Example 9

### IL-15 Promotes T Cell Survival and Inhibits T Cell Exhaustion

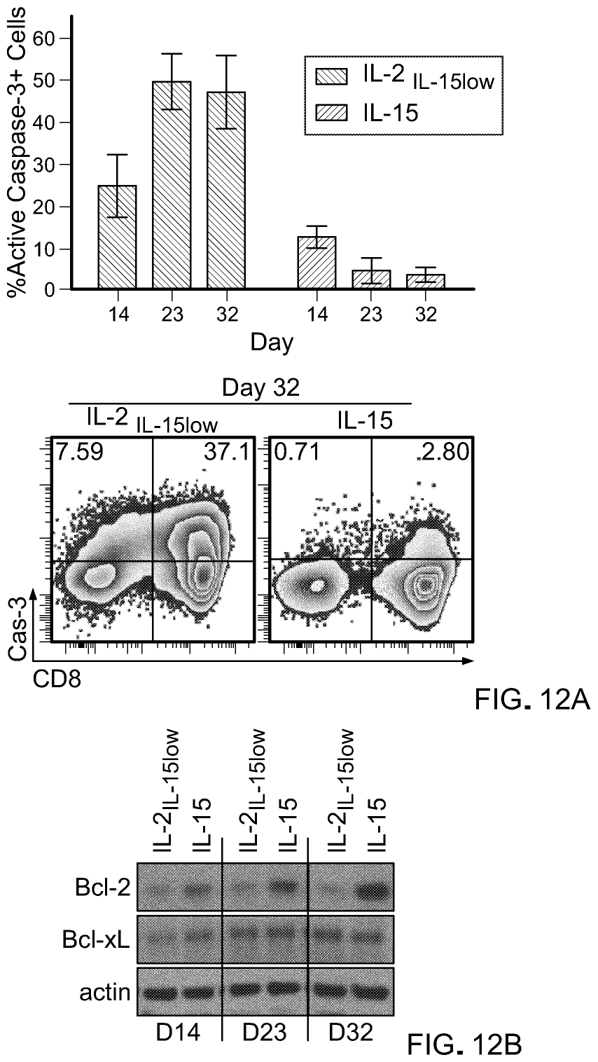

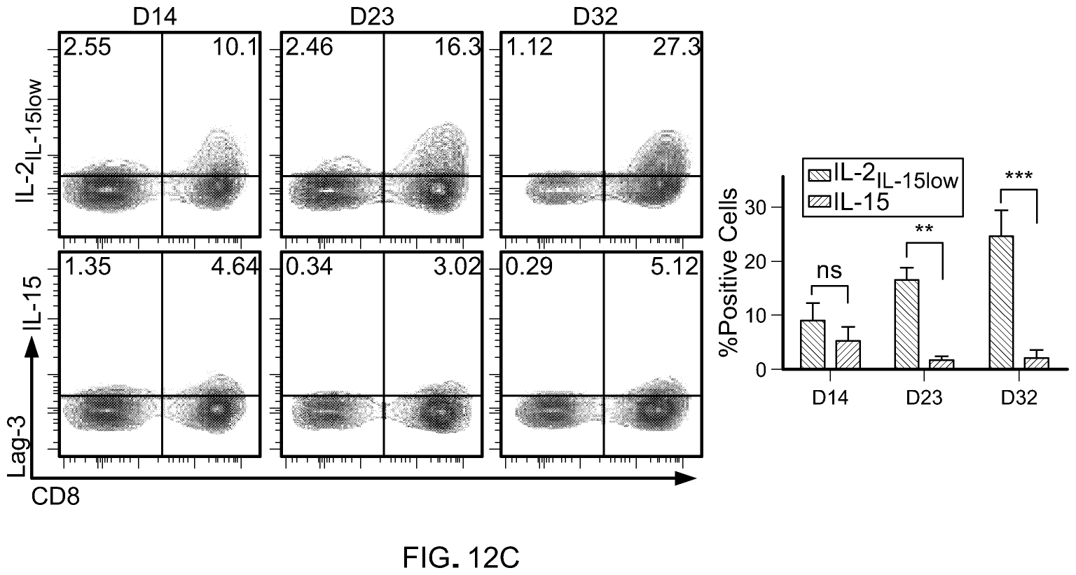

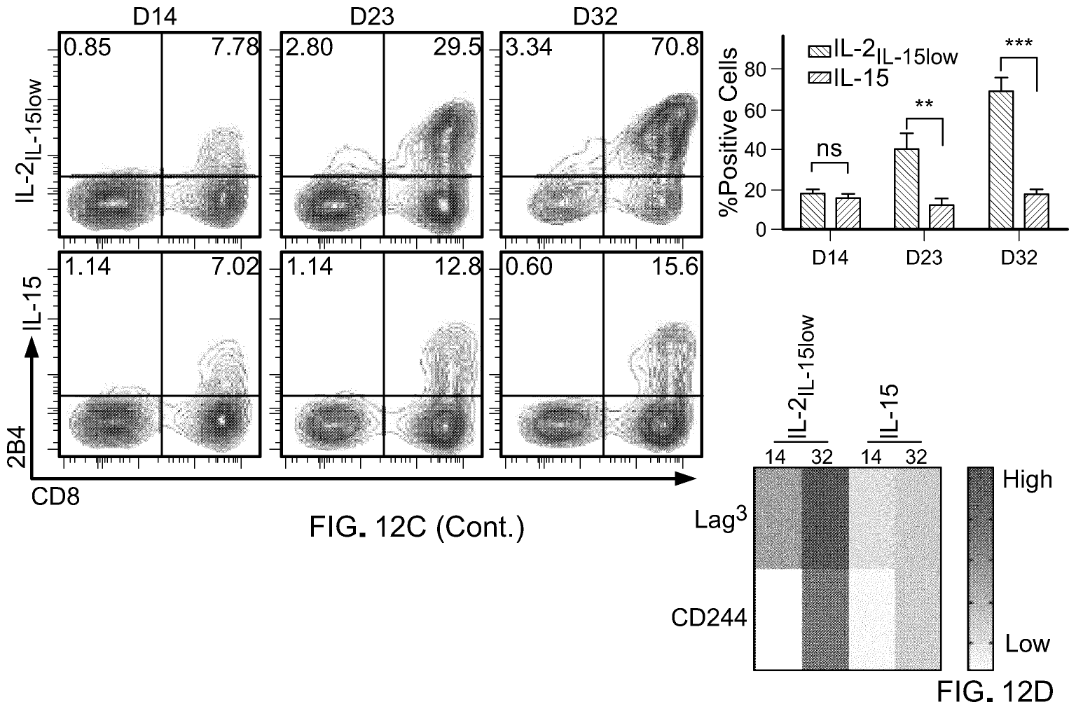

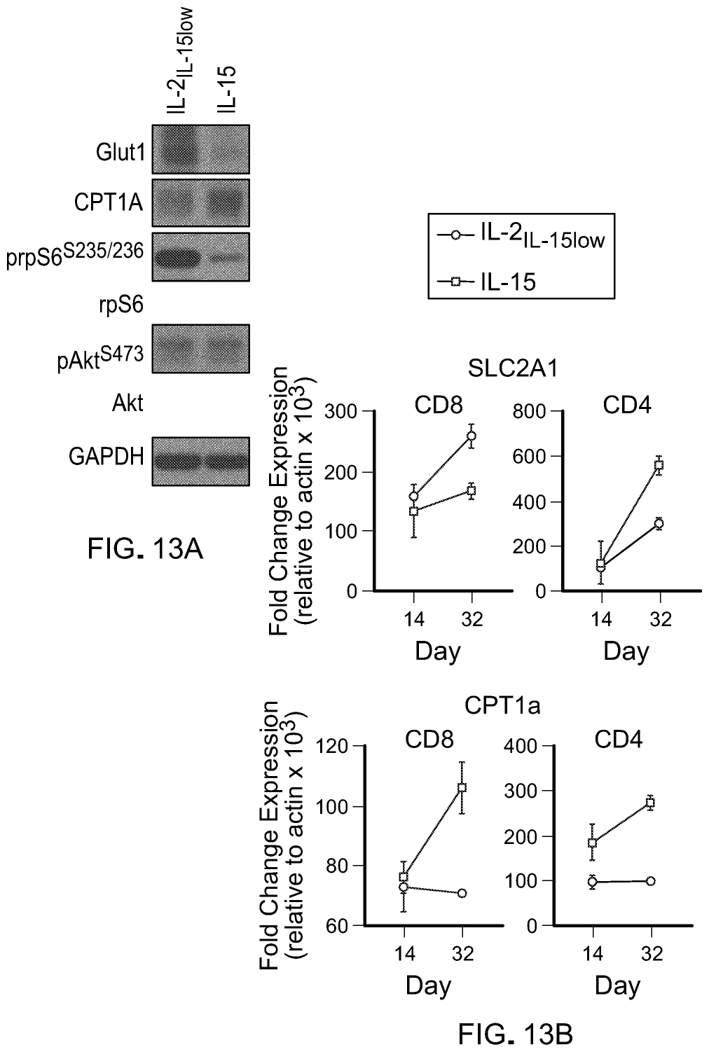

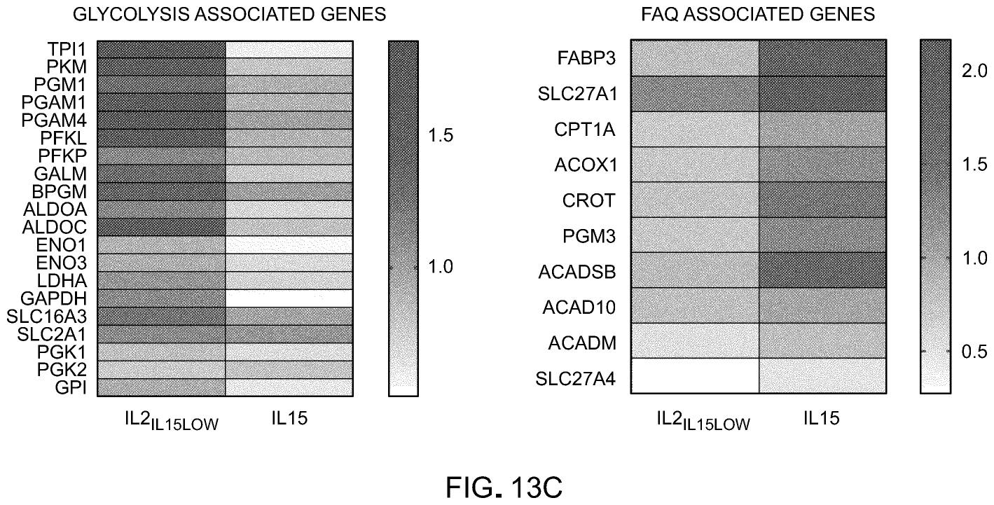

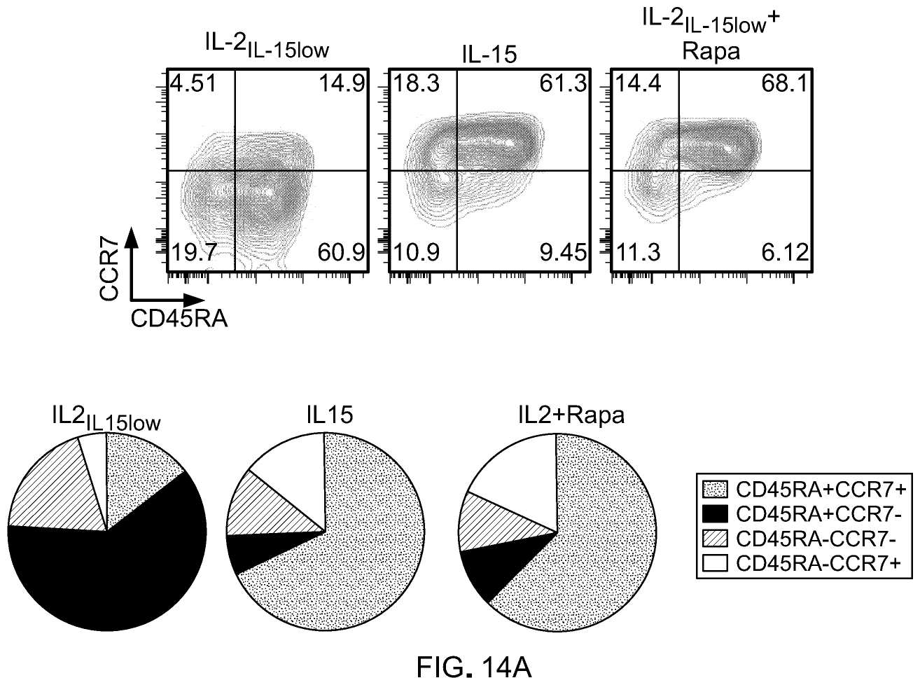

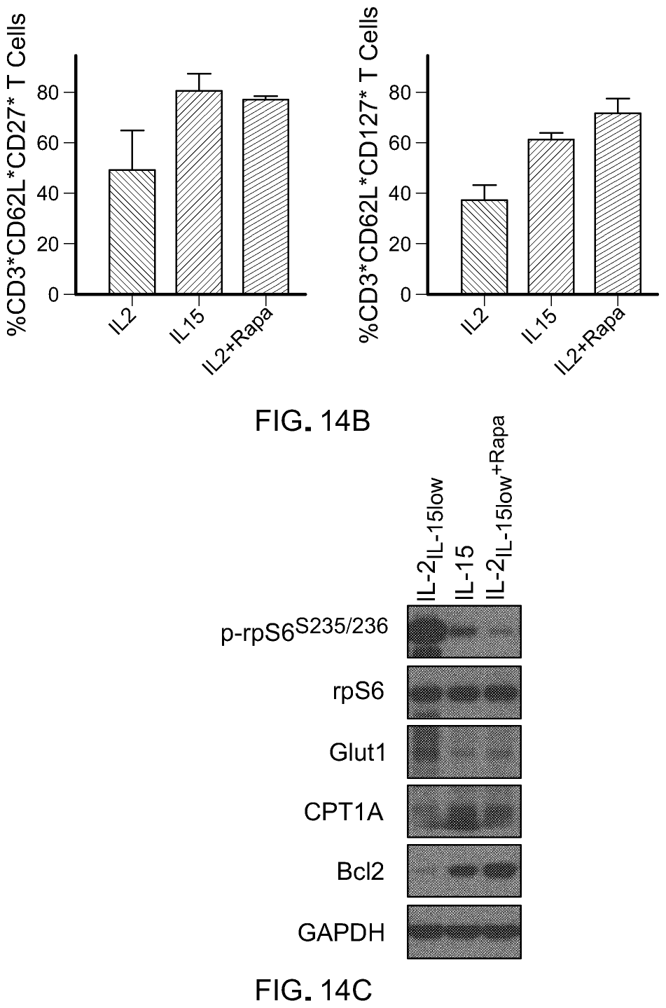

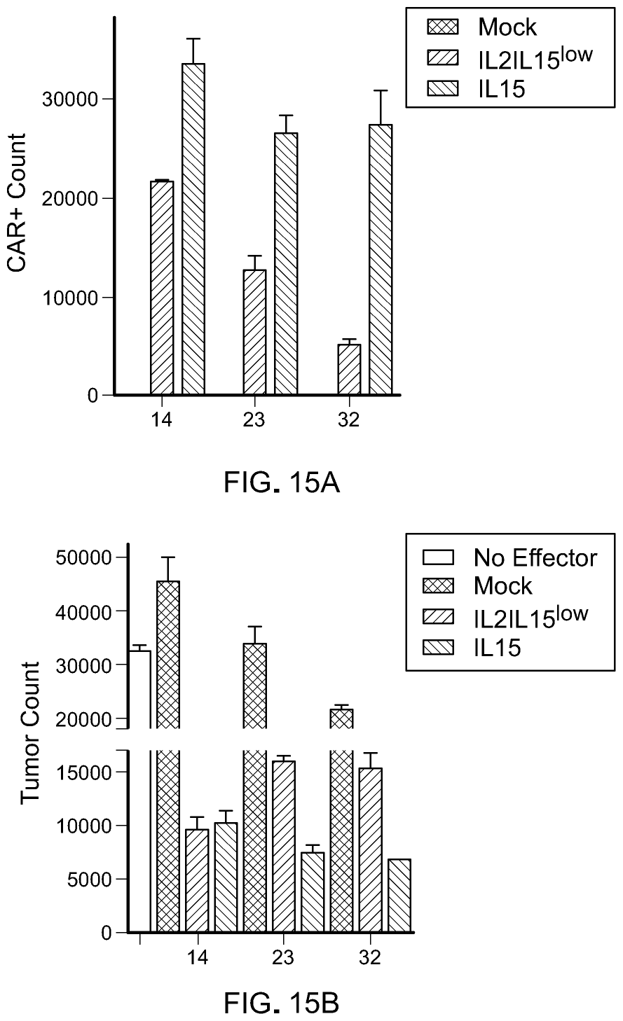

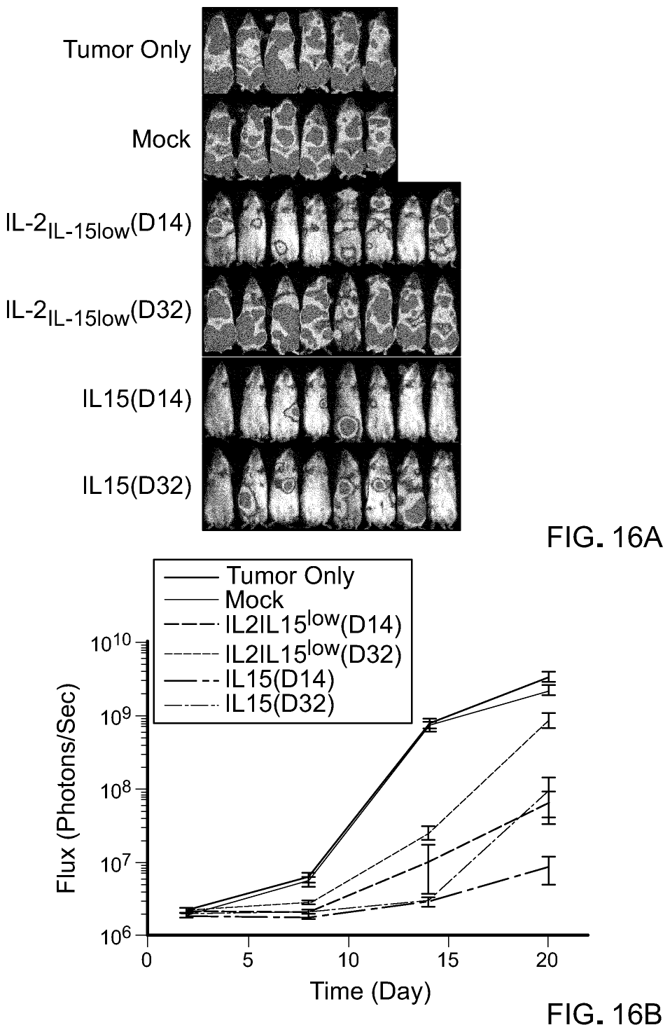

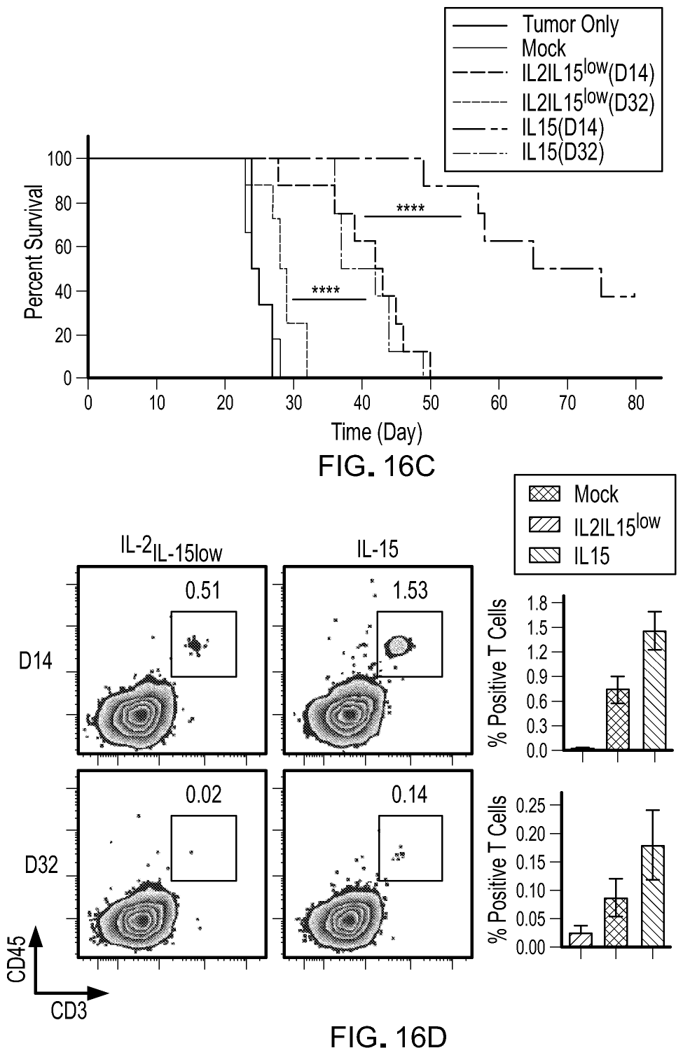

To further investigate additional factors that may impact the persistence of CAR T cells, we evaluated CAR T cell survival over extended culture conditions. Interestingly, recent data have indicated that caspase-3 activity is inhibited by IL-15-mediated posttranslational modifications. Our data indicate that IL-15-cultured T cells expressed significantly lower levels of active caspase-3 compared to IL-2IL-15low-generated T cells (FIG. 12(A)). Up-regulation of the anti-apoptotic molecule Bcl2 in IL-15-cultured T cells compared with IL-2IL-15low-generated T cells further confirms that IL-15 exerts an anti-apoptotic effect on T cells, which may improve their persistence in vivo (FIG. 12(B)).

Up-regulation of inhibitory receptors in CART cells negatively impacts their function and results in T cell exhaustion, which corresponds to up-regulation of receptors such as PD1, Lag3 and 2B4 and down-regulation of CD127 and accompanied by failure to self-renew. In our studies we demonstrate increased Lag3+ and 2B4+ cells in IL-21L-15low condition as compared with IL-15 alone that corresponded to increase in Lag3 and CD244 (2B4) gene expression in CD8+ population (FIG. 12(C) and FIG. 12(D)). Of note, in CD4+ cells, there was an overall higher expression of genes encoding for inhibitory molecules such as CTLA4 (ctla4), PD1 (pdcd1), Lag3 (lag3), PDL-1 (cd274) and suppressive factors such as IL13 and FOXP3 in IL-2IL-15low-cultured T cells (FIG. 11(C)).

### Example 10

### IL-15-Cultured CAR T Cells Exhibit Reduced mTORC1 Activity with Significant Reduction in Expression of Glycolytic Enzymes

We next sought to identify modifications in signaling pathways that may explain the phenotypical and functional differences observed in cells generated in different cytokine conditions. Multiple signal transduction pathways have been implicated in regulating cell differentiation and preserving memory stem cell phenotype. While Akt plays a major role in T cell effector differentiation, our results indicated that phosphorylation of Akt (pAkt) was not different in IL-2IL-15low-cultured T cells compared with IL-15-cultured cells. However, a significant decrease in mTORC1 activity as measured by reduced phosphorylation of the ribosomal protein S6 (rpS6) (FIG. 13(A)) was detected. mTORC1 signaling pathway has been involved in metabolic changes and regulation of glucose transport [8]. mTOR signaling increases glycolysis by increasing GLUT1 (slc2a1) expression and stimulating glycolytic enzyme activity. Consistent with this observation, IL-15-generated T lymphocytes exhibited enhanced expression of CPT1a (cpt1a), key enzyme regulating fatty acid oxidation and a slight decrease in Glut1 expression (FIG. 13(A)). To determine if decreased mTORC1 activity in IL-15-cultured T cells leads to commensurate changes in expression of genes in metabolism of T cells, we evaluated expression of key enzymes involved in glycolysis and fatty acid oxidation pathways. Interestingly, T cells cultured in IL-15 had markedly reduced expression of glycolytic enzymes and conversely increased expression of enzymes involved in fatty acid oxidation pathway (FIG. 13(B) and FIG. 13(C)).

Lastly, to determine if IL-15-mediated reduction of mTORC1 activity influences T cell phenotype and prevents T cell differentiation, cells were cultured with IL-2_{IL-15low}, IL-15 or IL-2_{IL-15low} plus rapamycin. Interestingly, IL-15 and IL-2_{IL-15low} plus rapamycin T cells exhibited very similar key naive/memory phenotype (FIG. 14(A) and FIG. 14(B)). Western blot analysis confirms down-regulation of p-rpS6 and Glut1 and up-regulation of CPT1a and Bcl2 in both IL-15 and IL-2_{IL-15low} plus rapamycin cultured cells (FIG. 14(C)). Collectively, these data suggest that IL-15-mediated reduction in mTORC1 activity may lead to decreased glycolysis and ultimately prevent T cell differentiation.

### Example 11

### IL-15-Cultured T Cells Exhibit Enhanced Proliferative Capacity that Correlates with Superior Antitumor Activity and Present In Vivo

The phenotypic and functional differences observed in T cells propagated in IL-15 compared to IL-2_{IL-15low} strongly suggested that IL-15 preserved the stem-like properties of T cells. One of the key characteristics of T cells with stem-like properties is their ability to self-renew and to differentiate into specialized cell types. We thus sought to compare the self-renewal and multipotency capacity of both cytokine conditions. In a prolonged in vitro killing assay, upon tumor antigen stimulation, IL-15-cultured T cells exhibited 1.5 fold higher self-renewal capacities compared to IL-2_{IL-15low} while maintaining their in vitro killing capacity over time (FIG. 15(A) and FIG. 15(B)).

Lastly, in order to highlight the phenotypic impact of CAR T cells on antitumor activity in vivo, CART cells expanded in IL-2_{IL-15low} or IL-15 for 14 or 32 days were administered to mice bearing Raji tumors. The CAR T cells maintained in culture for 32 days in presence of IL-2_{IL-15low} mediated minimum antitumor responses. However, the adoptive transfer of IL-15-generated CART cells promoted significantly more survival advantage (FIG. 16(A), FIG. 16(B) and FIG. 16(C)). Of note, the antitumor activity of IL-15-cultured T cells was not attributed to a specific CAR or donor type as similar antitumor activity was observed in IL13Ra2-targeted glioma models (data not shown). Consistent with our overall survival data, CART cells generated with IL-15 persisted in vivo significantly longer that CART cells generated in IL-21L-15low conditions. Notably IL-2IL-15low-generated CAR T cells exhibited a short half-life and were undetectable 7 days after infusion (FIG. 16(D)).

What is claimed is:

1. A method for expanding T cells, comprising:
(a) providing a population of human T cells; and
(b) culturing the population of human T cells for at least one day in a culture medium comprising exogenously added IL-15 at a concentration of at least 5 ng/ml.

2. The method of claim 1, wherein the culture medium comprises exogenously added IL-2 at a concentration of less than 50 U/ml.

3. The method of claim 1 or claim 2, wherein the population of human T cells is cultured for at least 5 days in the culture medium.

4. The method of claim 1 or claim 2, wherein exogenously added IL-15 is present at a concentration of at least 10 ng/ml.

5. The method of claim 1 or claim 2, wherein the population of human T cells comprises T cells expressing a CAR.

6. The method of claim 1 or claim 2, wherein the population of T cells comprises tumor infiltrating lymphocytes.

7. The method of claim 1 or claim 2, wherein the population of T cells is engineered to express a T cell receptor.

8. The method of claim 1 or claim 2, wherein the culture media comprises exogenously added IL-2 at a concentration of less than 10 U/ml.

9. The method of claim 1 or claim 2, wherein the culture media comprises exogenously added IL-2 at a concentration of less than 1 U/ml.

10. The method of claim 1 or claim 2, wherein the culture medium comprises exogenously added IL-7 at concentration of less than 5 ng/ml

11. The method of claim 1 or claim 2, wherein the culture medium comprise exogenously added IL-21 at concentration of less than 5 ng/ml.

12. The method of claim 1 or claim 2, wherein the culture medium comprises exogenously added IL-7 at concentration of less than 5 ng/ml and exogenously added IL-21 at concentration of less than 5 ng/ml.

13. The method of claim 1 or claim 2, wherein the culture medium comprises exogenously added IL-7 at concentration of less than 1 ng/ml

14. The method of claim 1 or claim 2, wherein the culture medium comprise exogenously added IL-21 at concentration of less than 1 ng/ml.

15. The method of claim 1 or claim 2, wherein the culture medium comprises exogenously added IL-7 at concentration of less than 1 ng/ml and exogenously added IL-21 at concentration of less than 1 ng/ml.

16. The method of claim 1 or claim 2, wherein the culture medium comprises no exogenously added IL-7.

17. The method of claim 1 or claim 2, wherein the culture medium comprises no exogenously added IL-21.

18. The method of claim 1 or claim 2, wherein the culture medium comprises no exogenously added IL-7 and no exogenously added IL-21.

19. The method of claim 1 or claim 2, wherein the population of cells is cultured in the culture medium for at least five days and less than 40 days.

20. The method of claim 1 or claim 2, wherein the population of cells is cultured in the culture medium for at least five days and less than 30 days.

21. The method of claim 1 or claim 2, wherein the population of cells is cultured for a period of time sufficient to expand the population less than 100-fold.

22. The method of claim 1 or claim 2, further comprising, prior to culturing the population of cells for at least one day in a culture medium comprising IL-15, culturing the population of cells in the presence of antibodies targeted to human CD3 and antibodies targeted to human CD28.

23. The method of claim 22 wherein the antibodies are present on a solid support.

24. The method of claim 1 or claim 2, wherein at least 30% of the T cells in the provided population of T cells are CD4+ and at least at least 10% of the T cells in the provided population of T cells are CD8+.

25. The method of claim 1 or claim 2, wherein at least 70% of the cells in the provided T cell population are CD4+ T cells.

26. The method of claim 1 or claim 2, wherein at least 70% of the cells in the provided T cell population are CD8+ T cells.

27. The method of claim 1 or claim 2, wherein at least 90% of the cells in the provided T cell population are CD4+ T cells.

28. The method of claim 1 or claim 2, wherein at least 90% of the cells in the provided T cell population are CD4+ T cells.

29. The method of claim 1 or claim 2, wherein at least 25% of the T cells in the provided population of T cells are CD45RA+.

30. The method of claim 1 or claim 2, wherein at least 50% of the T cells in the provided population of T cells are CD62L+.

31. The method of claim 1 or claim 2, wherein no more than 50% of the T cells in the provided population of T cells are CD62L−.

32. The method of claim 1 or claim 2, wherein the concentration of exogenously added IL-15 in the culture medium is no more than 100 ng/ml, 90 ng/ml, 80 ng/ml, 70 ng/ml. 60 ng/ml, 50 ng/ml, 40 ng/ml, 30 ng/ml, 20 ng/ml, or 15 ng/ml.

33. The method of claim 1 wherein the provided population of transduced human T cells are prepared by a method comprising obtaining a sample of PBMC from a human patient, treating the obtained PBMC to isolate a population of cells enriched for central memory T cells; memory stem T cells, and naïve T cells, and transducing at least a portion of the isolated population of cells to with a viral vector comprising an expression cassette encoding a chimeric antigen receptor.

34. The method of claim 33 wherein the step of treating the sample of PBMC to isolate a population of cells enriched for central memory T cells; memory stem T cells, and naïve T cells comprises: depleting the sample of PBMC of cells expressing CD14 and cells expressing CD25 and enriching for cells expressing CD62L to create a population of cells comprising: central memory T cells;
memory stem T cells, and naïve T cells.

35. The method of claim 33 wherein the wherein the step of treating the sample of PBMC to isolate a population of cells enriched for central memory T cells;
memory stem T cells, and naïve T cells comprises method does not comprise depleting cells expressing CD45RA.

36. The method of claim 33 wherein the population of human T cells are autologous to the patient.

37. The method of claim 33 wherein the population of human T cells are allogenic to the patient.

38. The method of claim 1, wherein the step of providing a population of human T cells comprising: providing a population of T cells expressing a CAR and comprising central memory T cells; memory stem T cells, and naïve T cells (T_{CM/SCM/N} CAR expressing cells).

39. The method of claim 20, wherein greater than 40% of the T_{CM/SCM/N} CAR expressing cells are CD45RA+ and greater than 70% of the T_{CM/SCM/N} CAR expressing cells are CD62L+.

40. The method of any of the forgoing claims wherein the culture medium comprises no exogenously added IL-2.

41. The method of any of the forgoing claims wherein the culture medium comprises no exogenously added IL-2, no exogenously added IL-7 and no exogenously added IL-21.

42. The method of any of the forgoing claims wherein the population of provided T cells is at least 70% CD3+/CD62L+.

43. The method of claim 42, wherein the population of provided T cells is at least 50% CD45RA+ or CD45RO+.

44. The method of claim 42 or 43, wherein the population of provided T cells is less than 10% CD14+ and less than 10% CD25+.

45. The method of claim 1, wherein the culture medium comprises IL-2 at a concentration of less than 50 U/ml.

46. The method of claim 1 or claim 2, wherein the population of human T cells is cultured for at least 5 days in the culture medium.

47. The method of claim 1 or claim 2, wherein IL-15 is present at a concentration of at least 10 ng/ml.

48. The method of claim 1 or claim 2, wherein the population of human T cells comprises T cells expressing a CAR.

49. The method of claim 1 or claim 2, wherein the population of T cells comprises tumor infiltrating lymphocytes.

50. The method of claim 1 or claim 2, wherein the population of T cells is engineered to express a T cell receptor.

51. The method of claim 1 or claim 2, wherein the culture media comprises IL-2 at a concentration of less than 10 U/ml.

52. The method of claim 1 or claim 2, wherein the culture media comprises IL-2 at a concentration of less than 1 U/ml.

53. The method of claim 1 or claim 2, wherein the culture medium comprises IL-7 at concentration of less than 5 ng/ml

54. The method of claim 1 or claim 2, wherein the culture medium comprise IL-21 at concentration of less than 5 ng/ml.

55. The method of claim 1 or claim 2, wherein the culture medium comprises IL-7 at concentration of less than 5 ng/ml and IL-21 at concentration of less than 5 ng/ml.

56. The method of claim 1 or claim 2, wherein the culture medium comprises IL-7 at concentration of less than 1 ng/ml

57. The method of claim 1 or claim 2, wherein the culture medium comprises IL-21 at concentration of less than 1 ng/ml.

58. The method of claim 1 or claim 2, wherein the culture medium comprises IL-7 at concentration of less than 1 ng/ml and IL-21 at concentration of less than 1 ng/ml.

59. The method of claim 1 or claim 2, wherein the culture medium comprises no exogenously added IL-7 throughout 90% of the culturing period.

60. The method of claim 1 or claim 2, wherein the culture medium comprises no exogenously added IL-21 throughout 90% of the culturing period.

61. The method of claim 1 or claim 2, wherein the culture medium comprises no exogenously added IL-2 throughout 90% of the culturing period.

62. The method of claim 1 or claim 2, wherein the culture medium comprises no exogenously added IL-2 throughout 70% of the culturing period.

63. The method of claim 1 or claim 2, wherein the culture medium comprises no exogenously added IL-7 and no exogenously added IL-21 throughout 90% of the culturing period.
